(19) United States
(12) Patent Application Publication
(10) Pub. No.: US 2010/0247606 A1
(43) Pub. Date: Sep. 30, 2010

(54) INTRAOCULAR SUSTAINED RELEASE DRUG DELIVERY SYSTEMS AND METHODS FOR TREATING OCULAR CONDITIONS

(75) Inventors: Michael R. ROBINSON, Irvine, CA (US); James A. Burke, Santa Ana, CA (US); Hui Liu, Irvine, CA (US); Werhner C. Orilla, Anaheim, CA (US); Lon T. Spada, Walnut, CA (US); Scott Whitcup, Laguna Hills, CA (US); Alazar N. Ghebremskel, Irvine, CA (US); Patrick M. Hughes, Aliso Viejo, CA (US); Kun Xu, Laguna Niguel, CA (US); Marianne M. Do, Orange, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/411,250

(22) Filed: Mar. 25, 2009

(51) Int. Cl. A61F 2/00; A61K 31/215; A61K 31/165; A61K 9/14; A61K 31/4025; A61K 31/426; A61K 31/415; A61K 31/42

(52) U.S. Cl. 424/426; 514/530; 514/622; 424/501; 514/422; 514/369; 514/385; 514/376

(57) ABSTRACT

Biocompatible, bioerodible sustained release implants and microspheres for intracameral or anterior vitreal placement include an anti-hypertensive agent and a biodegradable polymer effective to treat an ocular hypertensive condition (such as glaucoma) by relapsing therapeutic amount of the anti-hypertensive agent over a period of time between 10 days and 1 year.

### BACKGROUND

The present invention relates to intraocular systems and methods for treating ocular conditions. In particular the present invention is directed to local administration of a sustained release drug delivery system (i.e. drug incorporating microspheres and/or implants) to the anterior chamber (i.e. intracameral administration) and/or to anterior vitreous chamber of the eye to treat aqueous chamber elevated intraocular pressure (i.e. a hypertensive condition), as can be symptomatic of glaucoma or glaucoma risk.

The drug delivery systems of our invention can be a drug containing implant (i.e. a single, monolithic sustained release drug delivery system) or implants or a plurality of drug containing microspheres (synonymously “microparticles”). The drug delivery system can be used therapeutically to treat an ocular disease or condition such as elevated intraocular pressure and/or glaucoma. Glaucoma is a disease of the eye characterized by increased aqueous chamber intraocular pressure (IOP). Untreated glaucoma can result in blindness. Glaucoma can be primary or secondary glaucoma. Primary glaucoma in adults (congenital glaucoma) may be either open-angle or acute or chronic angle-closure. Secondary glaucoma results from pre-existing ocular diseases such as uveitis, intraocular tumor or an enlarged cataract. Various hypertensive agents have been used to lower IOP and treat glaucoma. For example, certain prostaglandins and their analogs and derivatives, such as the PGF_{2α} derivative (sometimes referred to as prostaglandin F2α analogue) latanoprost, sold under the trademark Xalatan®, have been used to treat ocular hypertension and glaucoma. Intraocular prostaglandin and prostamide implants and microspheres are disclosed by, for example U.S. patent application Ser. Nos. 11/368,845; 11/303,462, 10/837,260, and 12/259,153. Of particular interest are Examples 1 to 5 at pages 36 to 47 of Ser. No. 12/259,153. Also of interest is U.S. application Ser. No. 11/952,938. Additionally, U.S. Pat. Nos. 5,972,326 and 5,965,152 are also of interest.

Conventional treatment of glaucoma is by daily application of eye drops containing an anti-hypertensive drug to reduce IOP. Often patient compliance rate for regular, daily use of eyedrops is low. See eg Nordstrom et al. AJO 2005; 140:598. Additionally, eye infection can result from improper eye dropper use. Therefore, there is a need for a long term (i.e. sustained release) treatment method for ocular hypertension that can be conveniently administered for example during a visit to the doctor's office. Hence, it would be advantageous to provide sustained release intraocular drug delivery systems (comprising implants and/or microspheres) for intraocular therapeutic use to treat elevated IOP and/or glaucoma.

### SUMMARY

The present invention meets this need and provides a sustained release intraocular drug delivery systems, processes for making the drug delivery systems and methods for treating ocular conditions using the drug delivery systems. The sustained release intraocular drug delivery system is in the form of an implant or microspheres which advantageously provide for extended release of one or more therapeutic anti-hypertensive agents (eg a prostaglandin or a prostamide, such as latanoprost).

Definitions

The following definitions are used herein.

“About” means plus or minus ten percent of the number, parameter or characteristic so qualified.

“Microsphere” and “microparticle” are used synonymously to refer to a small diameter or dimension (see below) device or element that is structured, sized, or otherwise configured to be administered intracameral. Microspheres or microparticles includes particles, micro or nanospheres, small fragments, microparticles, nanoparticles, fine powders and the like comprising a biocompatible matrix encapsulating or incorporating a therapeutic agent. Microspheres are generally biocompatible with physiological conditions of an eye and do not cause significant adverse side effects. Microspheres made and used as disclosed herein can be administered intracameral and used safely without disrupting vision of the eye. Microspheres have a maximum dimension, such as diameter or length, less than 1 mm. For example, microparticles can have a maximum dimension less than about 500 μm. Microspheres can also have a maximum dimension no greater than about 200 μm, and preferably have a maximum dimension greater than 30 μm to about 50 μm or to about 75 microns. An “implant” is a drug delivery device which is considerably larger than a microsphere, and whereas a plurality (i.e. hundreds or thousands)) of microspheres are administered to treat an ocular condition (such as glaucoma) usually only one to at most six implants are administered for the same purpose.

“Ocular region” or “ocular site” means any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior (aqueous) chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

“Ocular condition” means a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

“Biodegradable polymer” means a polymer or polymers which degrade in vivo, and wherein erosion of the polymer or polymers over time occurs concurrent with or subsequent to release of the therapeutic agent. The terms “biodegradable” and “bioerodible” are equivalent and are used interchangeably herein. A biodegradable polymer may be a homopolymer, a copolymer, or a polymer comprising more than two different polymeric units. The polymer can be a gel or hydrogel type polymer, PLA or PLGA polymer or mixtures or derivatives thereof.

“Therapeutically effective amount” means level or amount of agent needed to treat an ocular condition, or reduce or prevent ocular injury or damage without causing significant negative or adverse side effects to the eye or a region of the eye. In view of the above, a therapeutically effective amount of a therapeutic agent, such as a latanoprost, is an amount that is effective in reducing at least one symptom of an ocular condition.

Implants and microspheres within the scope of our invention can release an anti-hypertensive agent over a relatively long period of time, for example, for at least about one week or for example for between about two months and about six months, after intraocular (i.e. intracameral) administration of anti-hypertensive agent containing implant or microspheres. Such extended release times facilitate obtaining successful treatment results. Preferably the sustained release intraocular drug delivery system is administered either intracameral (that is into the aqueous chamber [also called the anterior chamber] of the eye) or into the anterior portion of the posterior chamber (also called the vitreous chamber) of the eye.

An embodiment of our invention is a pharmaceutical composition for intraocular use to treat an ocular condition. The composition can comprise a plurality of microspheres made of a bioerodible polymer, and an anti-hypertensive such as latanoprost, bimatoprost and travoprost and their salts, esters and derivatives, contained by the microspheres. The microspheres can comprise from about 1% to about 99% by weight of the polymer and the polymer can be a PLGA and/or PLA. Additionally, the microspheres can have an average greatest dimension in a range of from about 5 microns to about 1 mm, for example the microspheres can have a mean diameter between about 15 microns and about 55 microns and the therapeutic agent can comprise from about 0.1% to about 90% by weight of the microspheres, such as between about 8 to 15 weight % latanoprost.

In another embodiment of our invention the composition can include a high viscosity hyaluronic acid and the ocular condition treated can be glaucoma. A detailed embodiment of our invention is a pharmaceutical composition for intraocular use to treat glaucoma comprising a plurality of microspheres made from a PLGA and/or PLA, latanoprost contained by the microspheres, and a high viscosity hyaluronic acid. Another embodiment of our invention is a pharmaceutical composition for intraocular use to treat glaucoma, the composition comprising a sustained release implant made from a PLGA polymer, a PLA polymer, and a PEG co-solvent, and; latanoprost contained by the implant, wherein the implant comprises about 30 weight percent latanoprost and the implant can release the latanoprost over a period of time of at least 20, 30, 40, 50, 60, 70 or up to 180 days.

Another embodiment of our invention is a method of treating glaucoma, the method comprising intraocular administration to a patient with glaucoma of a pharmaceutical composition comprising the implant set forth above or a plurality of microspheres made from a PLGA and/or PLA; latanoprost or an anti-hypertensive EP2 agonist contained by the microspheres or implant, and a high viscosity hyaluronic acid (HA), thereby treating the glaucoma. Preferably, the HA is used with the plurality of microspheres formulation but not with the single implant administered. The microspheres can release the anti-hypertensive agent for at least about one week after the administration step. The intraocular administration step can be carried out by injection into the sub-tenon space, such as into the anterior sub-tenon space and the pharmaceutical composition treats glaucoma by reducing baseline intraocular pressure by up to 20%, 30%, 40% or up to 50% or more.

Our invention encompasses a method for treating elevated intraocular pressure by intracameral administration to a patient with elevated intraocular pressure a plurality of sustained release biodegradable microspheres having an average diameter between 30 and 60 microns, the microspheres comprising from about 10 to about 30 weight percent an anti-hypertensive agent and from about 70 to about 90 weight percent a biodegradable polymer, wherein the microspheres release therapeutically effective amounts of the anti-hypertensive agent for a period of time between about 10 day and about 120 days. The biodegradable polymer can comprise a polylactic polyglycolic copolymer (PLGA) and/or a poly lactic acid polymer (PLA). The anti-hypertensive agent can be latanoprost, bimatoprost and travoprost and their salts, esters and prodrugs. Alternately, the anti-hypertensive agent can be one or more of the Compounds A to O (which are EP2 receptor agonists) (shown below), as well as their salts, esters and prodrugs:

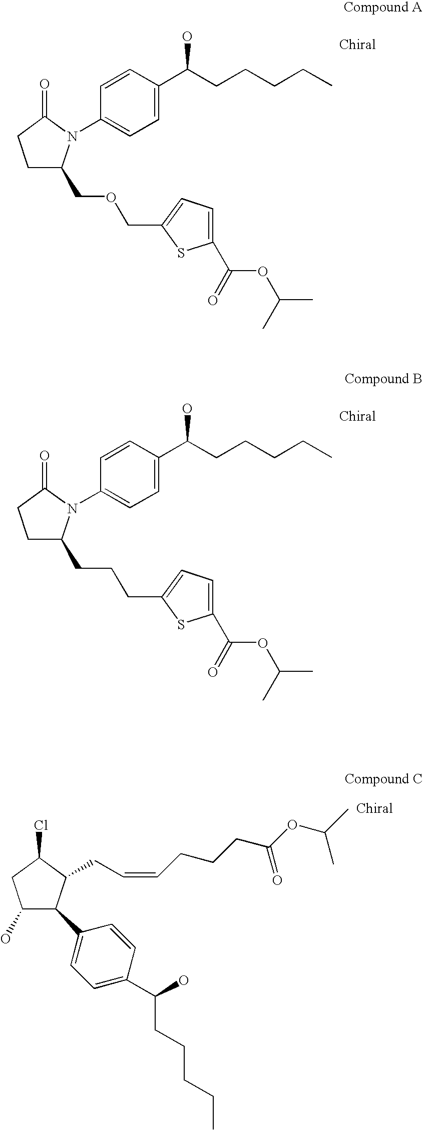

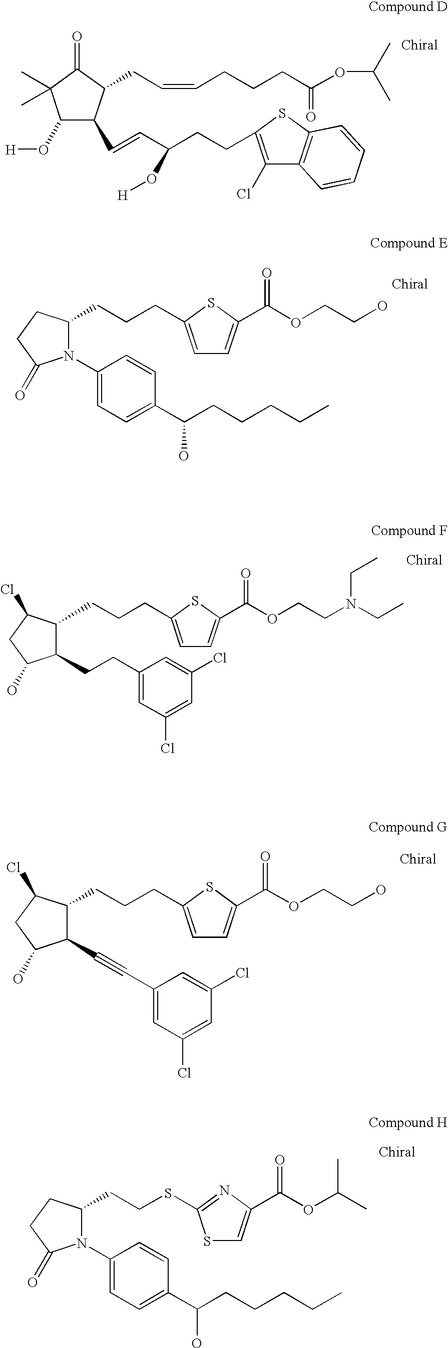

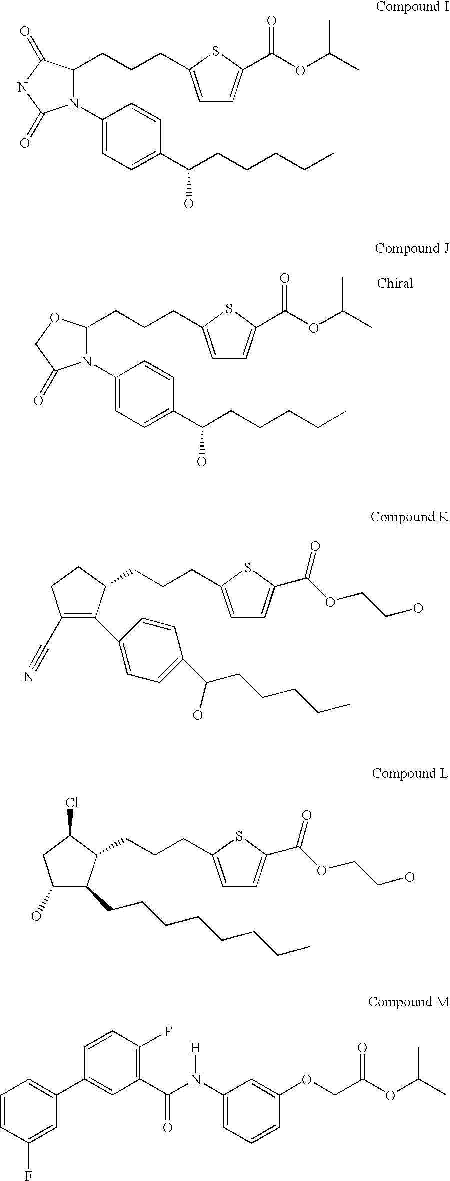

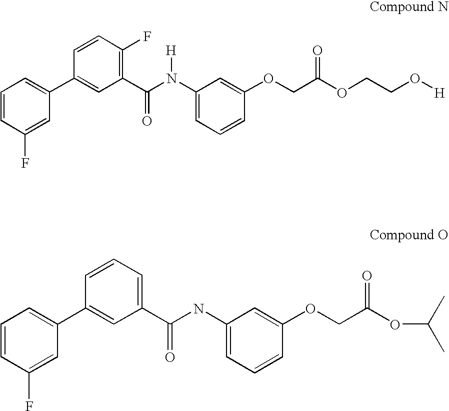

An embodiment of our invention includes as part of the drug delivery system a high viscosity hyaluronic acid. A detailed embodiment of our invention is a method for treating elevated intraocular pressure by intracameral administration to a patient with elevated intraocular pressure a plurality of sustained release biodegradable microspheres having an average diameter between 30 and 60 microns, the microspheres comprising from about 10 to about 30 weight percent latanoprost and from about 70 to about 90 weight percent a biodegradable polymer, wherein the microspheres release therapeutically effective amounts of the latanoprost for a period of time between about 10 day and about 120 days.

A further detailed embodiment of our invention is a method for treating elevated intraocular pressure by intracameral administration against the trabecular meshwork, to a patient with elevated intraocular pressure, a sustained release rod shaped implant comprising latanoprost and a biodegradable polymer, wherein the implant comprises from about 10 to about 50 weight percent an anti-hypertensive agent and from about 50 to about 90 weight percent a biodegradable polymer, wherein the implant releases therapeutically effective amounts of the latanoprost for a period of time between about 10 day and about 120 days.

Our invention also includes a pharmaceutical composition for intraocular use to treat an ocular condition, the composition comprising a plurality of sustained release biodegradable microspheres having an average diameter between 30 and 60 microns, the microspheres comprising from about 10 to about 30 weight percent an anti-hypertensive agent and from about 70 to about 90 weight percent a biodegradable polymer, wherein the microspheres release therapeutically effective amounts of the anti-hypertensive agent for a period of time between about 10 day and about 120 days. The microspheres can comprise from about 1% to about 99% by weight of the polymer.

A most preferred embodiment of our invention is an intracameral placed, sustained release, rod shaped, single, monolithic (i.e. the anti-hypertensive drug is homogenously distributed [i.e. reservoir type implants are excluded from the scope of the most preferred embodiment of our invention] throughout the polymeric matrix of the implant) implant (containing a therapeutic amount of an anti-hypertensive drug) which is about 2 mm to about 4 mm long and about 0.5 mm to 2 about mm wide, implanted at the 6 o'clock or at the 12 o'clock position against the trabecular meshwork using a syringe type (i.e. 22 gauge) applicator (injector). A disc shape implant is not preferred because it will not abut well and/or will not remain in place next to the trabecular meshwork. Placement against the trabecular meshwork of a small rod-shaped implant (with the dimensions given above) takes advantage of the aqueous chamber currents and the drawing of fluid into the trabecular meshwork to hold the implant in place against the trabecular meshwork, thereby preventing the implant from floating away out of it's placement position. With this most preferred embodiment there is no vision obscuration upon stable placement of the implant and no iris chafing.

Additional aspects and advantages of the present invention are set forth in the following description and claims, particularly when considered in conjunction with the accompanying drawings.

### DRAWINGS

FIG. 1 is a cross-sectional representation of an area of a normal human eye anterior chamber angle showing flow direction of the aqueous humor (horizontal arrows) through the large pores in the trabeculum to the juxtacannalicular area (indicated by the vertical arrow).

FIG. 2 is a schematic drawing in which the arrows show aqueous humor convection currents in the anterior chamber with microspheres indicated placed inferiorly in the anterior chamber.

FIG. 3A is an external photograph of a rabbit eye in primary gaze.

FIG. 3B is an image of the rabbit eye in 3A with fluorescein filters in place on a Heidelberg HRA imaging device two days after implantation of a fluorescein implant (arrow).

FIG. 3C is an external photograph of the rabbit eye rotated down.

FIG. 3D is an image of the rabbit eye in 3C with the HRA seven days after implantation of the fluorescein implant showing distribution (arrow) of fluorescein released from the implant.

FIG. 4 is a graph showing cumulative % of latanoprost released (y axis) in vitro (PBS with 0.1% triton) over time in days (x axis) from Formulation A microspheres.

FIG. 5 is a graph showing cumulative % of latanoprost released (y axis) in vitro (PBS with 0.1% triton) over time in days (x axis) from Formulation B microspheres.

FIG. 6 is a graph on the y axis percent change from baseline IOP and on the x axis time in days after drug delivery device intraocular administration. The FIG. 6 results were obtained after intracameral dog (Beagle) injection of Formulation A sustained release microspheres in the left dog eye (solid line in FIG. 6: “API”). The fellow (right) control eye (dashed line in FIG. 6: “Control”) showed no IOP reduction.

FIG. 7 is a graph of percent change from subject dog eye baseline intraocular pressure (y axis) against time in days (x axis) over the 84 day period after intracameral administration of the Example 5 bimatoprost bar shaped implant (“API”), showing that an IOP drop of about 50% to 60% was maintained through the 84 day observation period. The fellow (left or “control”) eye received a placebo (no bimatoprost) implant.

### DESCRIPTION

Transscleral delivery includes ocular topical (i.e. eyedrops) as well as intrascleral (i.e. subconjunctival or sub-Tenon) placement (eg by injection, insertion or implantation) placement drug administration. Our invention is based on the observation that transscleral delivery is an inefficient method for administering an anti-hypertensive agent (drug or biologic) to an aqueous chamber or vitreous chamber target tissue for the treatment of elevated intraocular pressure. We believe this to be so because of there are apparently three types of barriers hindering transscleral drug delivery—static, dynamic, and metabolic barriers. The ocular tissues that pose a physical barrier to drug diffusion (sclera, choroid-Bruch's membrane, retinal pigment epithelium) compromise the static barriers. Dynamic barriers are created by drug clearance mechanisms through blood and lymphatic vessels principally located in the conjunctiva, bulk fluid flow from anterior to posterior through the retina and clearance via the choriocapillaris and sclera, and transporter proteins of the retinal pigment epithelium. Metabolic barriers also exist in the eye, and reduce drug penetration into the eye by rapid degradation of scleral administered drugs. The dynamic barriers appear to be the most important barrier to transcleral (i.e. sub-Tenon's) delivery of therapeutic agents to the front of the eye (anterior chamber) for treating ocular hypertension and glaucoma.

Our invention is based on the discovery that direct intracameral or anterior intravitreal administration of the sustained release intraocular drug delivery system as set forth herein (comprising an anti-hypertensive agent containing implants or microspheres) can be effective use to treat an ocular condition, such as glaucoma, characterized by elevated intraocular pressure glaucoma by bypassing the robust scleral drug clearance mechanisms.

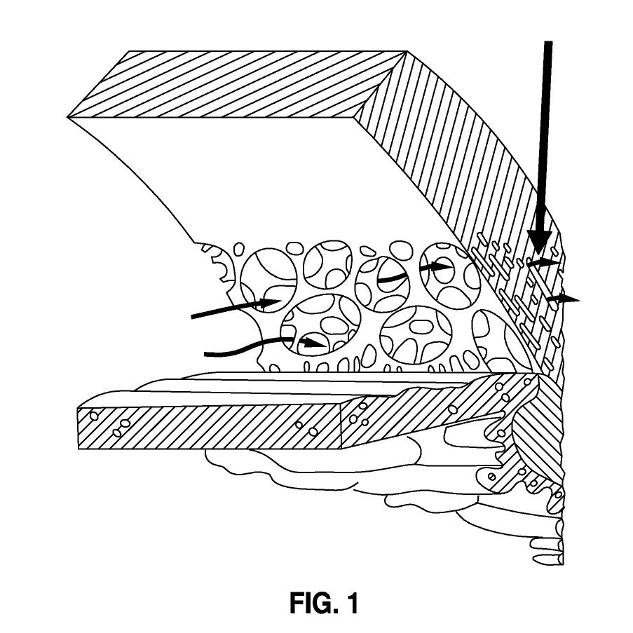

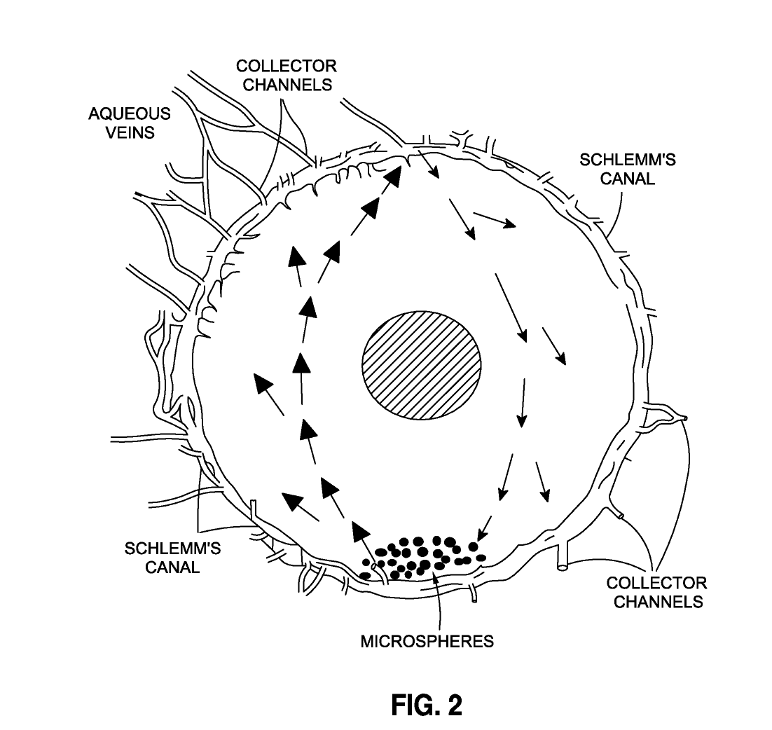

We determined existence of suitable alternative locations to deliver drugs to the front of the eye (anterior chamber) to lower the intraocular pressure (IOP) and evade the aggressive clearance of the transscleral barriers. Intracameral injections (i.e. direct injection into the anterior chamber) and anterior vitreous injections through the pars plana effectively avoid the transscleral barriers and improve the efficacy of the ocular anti-hypertensive compounds. Importantly, we discovered that intracameral drug delivery systems required development of new sustained released drug delivery system physical features, required for therapeutic efficacy because of the unique anatomy and physiology of the anterior chamber. For example, in the anterior chamber the aqueous flow rates are high and this can effectively eliminate sustained-release microspheres containing IOP lowering drugs and accelerate degradation of other polymeric delivery systems. Aqueous humor is secreted into the posterior chamber by the ciliary body, specifically by the non-pigmented epithelium of the ciliary body, through a process called ultrafiltration. It flows through the narrow cleft between the front of the lens and the back of the iris, to escape through the pupil into the anterior chamber. The aqueous humor drains 360 degrees into the trabecular meshwork that initially has pore size diameters ranging from 10 to under 30 microns in humans (see FIG. 1). FIG. 1 is a cross-sectional representational of the area of the eye anterior chamber angle showing the flow direction of the aqueous humor (horizontal arrows) through the larger pores (about 10 to less than 30 microns) in the trabeculum and gradually through to the juxtacannalicular area (indicated by the vertical arrow) where the pores reduce down to about 6 microns before entering Schlemm's canal. Aqueous humor drains through Schlemm's canal and exits the eye through 25 to 30 collector channels into the aqueous veins, and eventually into the episcleral vasculature and veins of the orbit (see FIG. 2). FIG. 2 is a schematic drawing in which the arrows indicate aqueous humor convection currents in the anterior chamber. Microspheres releasing anti-ocular hypertensive medication are shown placed inferiorly. Free drug eluting from the polymeric microspheres (or implant) enters the aqueous humor convection currents (arrows). The drug is then successfully dispersed throughout the anterior chamber and enters the target tissues such as the trabecular meshwork and the ciliary body region through the iris root region.

Another advantage of intracameral injection is that the anterior chamber is an immune privileged site in the body and less likely to react to foreign material, such as polymeric drug delivery systems. This is not the case in the sub-Tenon's space where the inflammatory reactions to foreign materials are common. In addition to the anterior chamber containing immunoregulatory factors that confers immune privilege, particles with diameters greater than 30 microns are less immunogenic and have a lower propensity towards causing ocular inflammation. Resident macrophages in the eye are the first line of defense with foreign bodies or infectious agents; however, particles larger than 30 microns are difficult to phagocytose. Therefore, particles larger than 30 microns are less prone to macrophage activation and the inflammatory cascade that follows.

We found that the efficiency of delivering drug to the aqueous humor with a polymeric release system is much greater with an intracameral location vs. sub-Tenon's application. Thus, less than 1% of drug delivered in the sub-Tenon's space will enter the aqueous humor whereas 100% of the drug released from an intracameral system will enter the aqueous humor. Therefore, it is expected that there will be lower drug loads required for effective intracameral drug delivery systems compared with sub-Tenon's and consequently, less systemic drug exposure. In addition, there will be less exposure of the conjunctiva to the active pharmaceutical ingredient, and less propensity towards developing conjunctival hyperemia when delivering drugs such as the prostaglandin analogues. Lastly, the drug will enter the conjunctival/episcleral blood vessel directly following an intracameral injections via the aqueous veins. This may minimize conjunctival hyperemia with prostaglandin analogues compared with a sub-Tenon's injection where numerous vessels are at risk of dilation with a high concentration of drug present diffusely in the extravascular space of the conjunctiva. Direct injections into the eye also obviate the need for preservatives, which when used in topical drops, can irritate the ocular surface.

Anti-hypertensive agents suitable for use as the active agent in the intracameral and intravitreal sustained release drug delivery systems disclosed herein include:
prostaglandins, prostamides and hypotensive lipids (e.g. bimatoprost (Lumigan]{bimatoprost increases uveoscleral outflow of aqueous humor as well as increases trabecular outflow} and the compounds set forth in U.S. Pat. No. 5,352,708). Prostaglandins are a class of pharmacologically active hormone like substances made in various mammalian tissues, which are derived from arachidonic acid, and mediate a wide range of physiological functions including blood pressure, smooth muscle contraction and inflammation. Examples of prostaglandins are prostaglandin E₁ (alprostadil), prostaglandin E₂ (dinoprostone), latanoprost and travoprost. Latanoprost and travoprost are actually prostaglandin prodrugs (i.e. 1-isopropyl esters of a prostaglandin) however, they are referred to as prostaglandins because they act on the prostaglandin F receptor, after being hydrolyzed to the 1-carboxylic acid. A prostamide (also called a prostaglandin-ethanolamide) is a prostaglandin analogue, which is pharmacologically unique from a prostaglandin (i.e. because prostamides act on a different cell receptor [the prostamide receptor] than do prostaglandins), and is a neutral lipid formed a as product of cyclo-oxygenase-2 (“COX-2”) enzyme oxygenation of an endocannabinoid (such as anandamide). Additionally, prostamides do not hydrolyze in-situ to the 1-carboxylic acid. Examples of prostamides are bimatoprost (the synthetically made ethyl amide of 17-phenyl prostaglandin F_{2α}) and prostamide F_{2α}.
prostaglandin analogues (prostaglandin analogues increase uveoscleral outflow of aqueous humor)(i.e. latanoprost [Xalatan], travoprost (Travatan), unoprostone;
EP2/EP4 receptor agonists;
beta-adrenergic receptor antagonists (such as timolol, betaxolol, levobetaxolol, carteolol, levobunolol, and propranolol, which decrease aqueous humor production by the ciliary body);
alpha adrenergic receptor agonists such as brimonidine (Alphagan) and apraclonidine (iopidine) (which act by a dual mechanism, decreasing aqueous production and increasing uveoscleral outflow);
less-selective sympathomimetics such as epinephrine and dipivefrin (Propine) (act to increase outflow of aqueous humor through trabecular meshwork and possibly through uveoscleral outflow pathway, probably by a beta 2-agonist action;
Miotic agents (parasympathomimetics) such as pilocarpine (acts by contraction of the ciliary muscle, tightening the trabecular meshwork and allowing increased outflow of the aqueous humor);
Carbonic anhydrase inhibitors such as dorzolamide (Trusopt), brinzolamide (Azopt), acetazolamide (Diamox) (lower secretion of aqueous humor by inhibiting carbonic anhydrase in the ciliary body)
Rho-kinase inhibitors (lower IOP by disrupting the actin cytoskeleton of the trabecular meshwork;
calcium channel blockers;
vaptans (vasopressin-receptor antagonists);
anecortave acetate and analogues;
ethacrynic acid;
cannabinoids;
beta-blockers (or .beta.-adrenergic antagonists) including carteolol, levobunolol, metiparanolol, timolol hemihydrate, timolol maleate, beta.1-selective antagonists such as betaxolol;
non-selective adrenergic agonists such as epinephrine borate, epinephrine hydrochloride, and dipivefrin;
alpha₂ selective adrenergic agonists such as apraclonidine and brimonidine;
Carbonic anhydrase inhibitors including acetazolamide, dichlorphenamide, methazolamide, brinzolamide, and dorzolamide;
Cholinergic Agonists including direct acting cholinergic agonists such as carbachol, pilocarpine hydrochloride; pilocarbine nitrate, and pilocarpine;
chlolinesterase inhibitors such as demecarium, echothiophate and physostigmine;
Glutamate antagonists;
Calcium channel blockers including memantine, amantadine, rimantadine, nitroglycerin, dextrophan, detromethorphan, dihydropyridines, verapamil, emopamil, benzothiazepines, bepridil, diphenylbutylpiperidines, diphenylpiperazines, fluspirilene, eliprodil, ifenprodil, tibalosine, flunarizine, nicardipine, nifedimpine, nimodipine, barnidipine, verapamil, lidoflazine, prenylamine lactate and amiloride;
Prostamides such as bimatoprost, or pharmaceutically acceptable salts or prodrugs thereof; and prostaglandins including travoprost, chloprostenol, fluprostenol, 13,14-dihydro-chloprostenol, isopropyl unoprostone, and latanoprost;
AR-102 (a prostaglandin FP agonist available from Aerie Pharmaceuticals, Inc.;
AL-3789 (anecortave acetate, an angiostatic steroid available from Alcon);
AL-6221 (travaprost [Travatan] a prostaglandin FP agonist;
PF-03187207 (a nitric-oxide donating prostaglandin available from by Pfizer)
PF-04217329 (also available from Pfizer);
INS115644 (a lantrunculin B compound available from Inspire Pharmaceuticals), and;
INS117548 (Rho-kinase inhibitor also available from inspire Pharmaceuticals).

Combinations of ocular anti-hypertensives, such as a beta blocker and a prostaglandin analogue, can also be used in the delivery systems. These include Ganfort (bimatoprost/timolol), Extravan or Duotrav (travoprost/timolol), Xalcom (latanoprost/timolol, Combigan (brimonidine/timolol, and Cosopt (dorzolamide/timolol). In combination with an IOP lowering drug, an agent that confers neuroprotection can also be placed in the delivery system and includes memantine and serotonergics [e.g., 5-HT.sub.2 agonists, such as S-(+)-1-(2-aminopropyl)-indazole-6-ol)].

We have developed implants and microspheres which can release drug loads over various time periods. These implants or microspheres, which when inserted intracameral or into the anterior vitreous therapeutic provide therapeutic levels of an anti-hypertensive agent for extended periods of time (e.g., for about 1 week up to about one year). Additionally, we have developed novel methods for making implants and microspheres. The anti-hypertensive agent of the present implants and microspheres is preferably from about 1% to 90% by weight of the microspheres. More preferably, the anti-hypertensive agent is from about 5% to about 30% by weight of the implant or microspheres. In a preferred embodiment, the anti-hypertensive agent comprises about 15% by weight of the microsphere (e.g., 5%-30 weight %). In another embodiment, the anti-hypertensive agent comprises about 40% by weight of the microspheres.

Suitable polymeric materials or compositions for use in the implant or microspheres include those materials which are compatible, that is biocompatible, with the eye so as to cause no substantial interference with the functioning or physiology of the eye. Such materials preferably are at least partially and more preferably substantially completely biodegradable or bioerodible.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present microspheres.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate. Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyvinyl alcohol, polyesters, polyethers and combinations thereof which are biocompatible and may be biodegradable and/or bioerodible. Some preferred characteristics of the polymers or polymeric materials for use in the present invention may include biocompatibility, compatibility with the selected therapeutic agent, ease of use of the polymer in making the drug delivery systems of the present invention, a half-life in the physiological environment of at least about 6 hours, preferably greater than about one day, and water insolubility.

The biodegradable polymeric materials which are included to form the matrix are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implants or microspheres. Different molecular weights of the same or different polymeric compositions may be included in the microspheres to modulate the release profile. For latanoprost implants, the relative average molecular weight of the polymer will preferably range from about 4 to about 25 kD, more preferably from about 5 to about 20 kD, and most preferably from about 5 to about 15 kD.

In some implants and microspheres, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the microspheres. The percentage of polylactic acid in the polylactic acid polyglycolic acid (PLGA) copolymer can be 0-100%, preferably about 15-85%, more preferably about 35-65%. In some implants, a 50/50 PLGA copolymer is used.

The implants and microspheres can be monolithic, i.e. having the active agent or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated microspheres may be of benefit in some circumstances, where the therapeutic level of the drug falls within a narrow window. In addition, the therapeutic component, including the latanoprost component, may be distributed in a non-homogenous pattern in the matrix. For example, the microspheres may include a portion that has a greater concentration of the latanoprost relative to a second portion of the microspheres.

The microspheres disclosed herein may have a size of between about 5 μm and about 1 mm, or between about 10 μm and about 0.8 mm for administration with a needle. For needle-injected microspheres, the microspheres may have any appropriate dimensions so long as the longest dimension of the microsphere permits the microsphere to move through a needle. This is generally not a problem in the administration of microspheres.

The total weight of implant or microsphere in a single dosage an optimal amount, depending on the volume of the anterior chamber and the activity or solubility of the active agent. Most often, the dose is usually about 0.1 mg to about 200 mg of implant or microspheres per dose. For example, a single intracameral injection may contain about 1 mg, 3 mg, or about 5 mg, or about 8 mg, or about 10 mg, or about 100 mg or about 150 mg, or about 175 mg, or about 200 mg of microspheres, including the incorporated therapeutic component.

The implant or microspheres may be of any particulate geometry including micro and nanospheres, micro and nanoparticles, spheres, powders, fragments and the like. The upper limit for the microsphere size will be determined by factors such as toleration for the implant, size limitations on insertion, desired rate of release, ease of handling, etc. Spheres may be in the range of about 0.5 μm to 4 mm in diameter, with comparable volumes for other shaped particles.

The proportions of the anti-hypertensive agent, polymer, and any other modifiers may be empirically determined by formulating several microsphere batches with varying average proportions. A USP approved method for dissolution or release test can be used to measure the rate of release (USP 23; NF 18 (1995) pp. 1790-1798). For example, using the infinite sink method, a weighed sample of the microspheres is added to a measured volume of a solution containing 0.9% NaCl in water, where the solution volume will be such that the drug concentration is after release is less than 5% of saturation. The mixture is maintained at 37° C. and stirred slowly to maintain the microspheres in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the therapeutic component, the implants and microspheres disclosed herein may include or may be provided in compositions that include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from about 0.001% to about 5% by weight and preferably about 0.01% to about 2% by weight. In at least one of the present microspheres, a benzylalkonium chloride preservative is provided in the implant, such as when the latanoprost consists essentially of bimatoprost

Various techniques may be employed to produce the implants and/or microspheres described herein. Useful techniques include, but are not necessarily limited to, self-emulsification methods, super critical fluid methods, solvent evaporation methods, phase separation methods, spray drying methods, grinding methods, interfacial methods, molding methods, injection molding methods, combinations thereof and the like.

As discussed herein, the polymeric component recited in the present method may comprise a biodegradable polymer or biodegradable copolymer. In at least one embodiment, the polymeric component comprises a poly (lactide-co-glycolide) PLGA copolymer. In a further embodiment, the PLGA copolymer has a lactide/glycolide ratio of 75/25. In a still further embodiment, the PLGA copolymer has at least one of a molecular weight of about 63 kilodaltons and an inherent viscosity of about 0.6 dL/g.

In addition, the present population of microparticles may have a maximum particle diameter less than about 200 μm. In certain embodiments, the population of microparticles has an average or mean particle diameter less than about 50 μm. In further embodiments, the population of microparticles has a mean particle diameter from about 30 μm to about 50 μm.

The anti-hypertensive agent containing implants and microspheres disclosed herein can be used to treat an ocular condition, such as the following:
maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema.
Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

A pharmaceutical composition (such as an implant or microspheres) within the scope of our invention can be formulated with a high viscosity, polymeric gel to reduce dispersion of the composition upon intraocular injection. Preferably, the gel has a high shear characteristic, meaning that the gel can be injected into an intraocular site through a 25-30 gauge needle, and more preferably through a 27-30 gauge needle. A suitable gel for this purpose can be a hydrogel or a colloidal gel formed as a dispersion in water or other aqueous medium. Examples of suitable gels include synthetic polymers such as polyhydroxy ethyl methacrylate, and chemically or physically crosslinked polyvinyl alcohol, polyacrylamide, poly(N-vinyl pyrolidone), polyethylene oxide, and hydrolysed polyacrylonitrile. Examples of suitable hydrogels which are organic polymers include covalent or ionically crosslinked polysaccharide-based hydrogels such as the polyvalent metal salts of alginate, pectin, carboxymethyl cellulose, heparin, hyaluronate (i.e. polymeric hyaluronic acid) and hydrogels from chitin, chitosan, pullulan, gellan, xanthan and hydroxypropylmethylcellulose. Commercially available dermal fillers (such as Hylafrom®, Restylane®, Sculptura™ and Radiesse) can be used as the high viscosity gel in embodiments of our pharmaceutical composition.

Hyaluronic acid (“HA”) is a polysaccharide made by various body tissues. U.S. Pat. No. 5,166,331 discusses purification of different fractions of hyaluronic acid for use as a substitute for intraocular fluids and as a topical ophthalmic drug carrier. Other U.S. patent applications which discuss ocular uses of hyaluronic acid include Ser. Nos. 11/859,627; 11/952,927; 10/966,764; 11/741,366; and 11/039,192 The pharmaceutical compositions within the scope of our invention preferably comprise a high viscosity hyaluronic acid with an average molecular weight between about 1 and 4 million Daltons, and more preferably with an average molecular weight between about 2 and 3 million Daltons, and most preferably with an average molecular weight of about (±10%) 2 million Daltons.

Dry uncrosslinked HA material comprises fibers or powder of commercially available HA, for example, fibers or powder of sodium hyaluronate (NaHA). The HA may be bacterial-sourced sodium hyaluronate, animal derived sodium hyaluronate or a combination thereof. In some embodiments, the dry HA material is a combination of raw materials including HA and at least one other polysaccharide, for example, glycosaminoglycan (GAG). In our invention the HA used comprises or consists of high molecular weight HA. That is, nearly 100% of the HA material in the present compositions is a high molecular weight HA. High molecular weight HA means HA with a molecular weight of at least about 1.0 million Daltons (mw≧10⁶ Da) to about 4.0 million Da (mw≦4×10⁶ Da). For example, the high molecular weight HA in the present compositions may have a molecular weight of about 2.0 million Da (mw 2×10⁶ Da). In another example, the high molecular weight HA may have a molecular weight of about 2.8 million Da (mw 2.8×10⁶ Da).

In an embodiment of our invention, dry or raw HA material (in this specific example, NaHA) having a desired high/low molecular weight ratio is cleaned and purified. These steps generally involved hydrating the dry HA fibers or powder in the desired high/low molecular weight ratio, for example, using pure water, and filtering the material to remove large foreign matters and/or other impurities. The filtered, hydrated material is then dried and purified. The high and low molecular weight NaHA may be cleaned and purified separately, or may be mixed together, for example, in the desired ratio, just prior to crosslinking. At this stage in the process, the pure, dried NaHA fibers are hydrated in an alkaline solution to produce an uncrosslinked NaHA alkaline gel. Any suitable alkaline solution may be used to hydrate the NaHA in this step, for example, but not limited to an aqueous solution containing NaOH. The resulting alkaline gel will have a pH above 7.5, for example, a pH above 8, for example, a pH above 9, for example, a pH above 10, for example, a pH above 12, for example, a pH above 13. In this specific example, the next step in the manufacturing process comprises the step of crosslinking the hydrated, alkaline NaHA gel with a suitable crosslinking agent, for example, BDDE.

The step of crosslinking may be carried out using means known to those of skill in the art. Those skilled in the art appreciate how to optimize the conditions of crosslinking according to the nature of the HA, and how to carry out the crosslinking to an optimized degree. In some embodiments of the present invention, the degree of crosslinking is at least about 2% to about 20%, for example, is about 4% to about 12%, wherein the degree of crosslinking is defined as the percent weight ratio of the crosslinking agent to HA-monomeric units in the composition. The hydrated crosslinked, HA gel may be neutralized by adding an aqueous solution containing HCl. The gel is then swelled in a phosphate buffered saline solution for a sufficient time and at a low temperature.

In certain embodiments, the resulting swollen gel (HA) is a cohesive gel having substantially no visible distinct particles, for example, substantially no visibly distinct particles when viewed with the naked eye. In some embodiments, the gel has substantially no visibly distinct particles under a magnification of less than 35×. The gel ((HA) is now purified by conventional means for example, dialysis or alcohol precipitation, to recover the crosslinked material, to stabilize the pH of the material and remove any unreacted crosslinking agent. Additional water or slightly alkaline aqueous solution can be added to bring the concentration of the NaHA in the composition to a desired concentration. In some embodiments, the concentration of NaHA in the composition is in a range between about 10 mg/ml to about 30 mg/ml.

Implants within the scope of our invention can be administered using any suitable intraocular injection device including the applicators (injectors) shown in U.S. patent application Ser. Nos. 11/455,392; 11/552,835; 11/552,630, and 12/355,709.

Embodiments of our invention can be sustained release biodegradable microspheres or implants. A preferred embodiment of our invention is a PLA and/or PLGA implant containing an anti-hypertensive agent because we have determined that implants of such composition result in significantly less inflammatory (i.e. less corneal hyperemia) upon intracameral or anterior vitreal administration. An embodiment of our invention can comprise a drug delivery system with a plurality of anti-hypertensive agents contained in different segments of the same implant or in different implants administered at the same time. For example one segment (i.e. one implant) can contain a muscarinic anti-hypertensive agent, a second segment (i.e. a second implant) can contain a anti-hypertensive prostaglandin and third segment (i.e. a third implant) can contain an anti-hypertensive beta blocker. Multiple implants (“segments”) can be injected simultaneously, for example, one implant with an anti-hypertensive agent to enhance aqueous outflow through the trabecular meshwork (e.g. a muscarinic agent), a second implant can be used to enhance uveoscleral flow (e.g. a hypotensive lipid), and a third implant can reduce aqueous humor production (e.g. a beta blocker). Multiple hypotensive agents with different mechanisms of action can be more effective at lowering IOP than monotherapy, that is use of a single type of an anti-hypertensive agent. Multiple segments (implants) have the advantage of permitting lower doses of each separate anti-hypertensive is agent used than the dose necessary with monotherapy, thereby reducing the side effects of each anti-hypertensive agent used. A separate and additional segment, containing for example a neuroprotective or neuroenhancing compound, can also be delivered with other segments containing anti-hypertensive agents.

When using multiple segments (i.e. a plurality of implants administered), each segment is preferably has a length no greater than about 2 mm. Preferably, the total umber of segments administered in the same a 22 to 25 G diameter needle bore is about four. With a 27 G diameter needle total segments length within the needle bore or lumen can be up to about 12 mm.

We determined that the trabecular meshwork (TM) has a detectable fluid uptake or suction action upon aqueous chamber fluid. This TM fluid uptake causes microspheres (MS) with a diameter of less than 30 microns to be drawn into the TM as we determined by gonioscopy imaging.

We also determined that the fluid uptake action of the TM can be exploited to keep MS or implants that have an appropriate geometry from floating around the anterior chamber causing visual obscuration. Gravity brings these implants down to the 6 o'clock position and we noted the implants or MS are very stable (relatively immobile) in this position. Implants that can be intraocular administered by a 22 G to 30 G diameter needle with lengths totaling no more than about 6 to 8 mm (all segments included) are most preferred to take advantage of the TM fluid uptake mechanism with resulting intraocular implant immobility and no visual obscuration. Thus despite being firmly in the 6 o'clock position in the anterior chamber due to TM fluid uptake effect, the implants can have release rates that exceed the TM clearance rate and this allows anti-hypertensive agent released by the implants to rapidly fill the anterior chamber and distribute well into the target tissues along a 360 degrees distribution pattern. Our examination of the implants in the angle of the anterior chamber with gonioscopy showed that the there was no encapsulation of nor inflammatory tissue in the vicinity of the implants.

### EXAMPLES

The following examples set forth non-limiting embodiments of our invention.

### Example 1

### Determination of Anterior Chamber Convection Currents

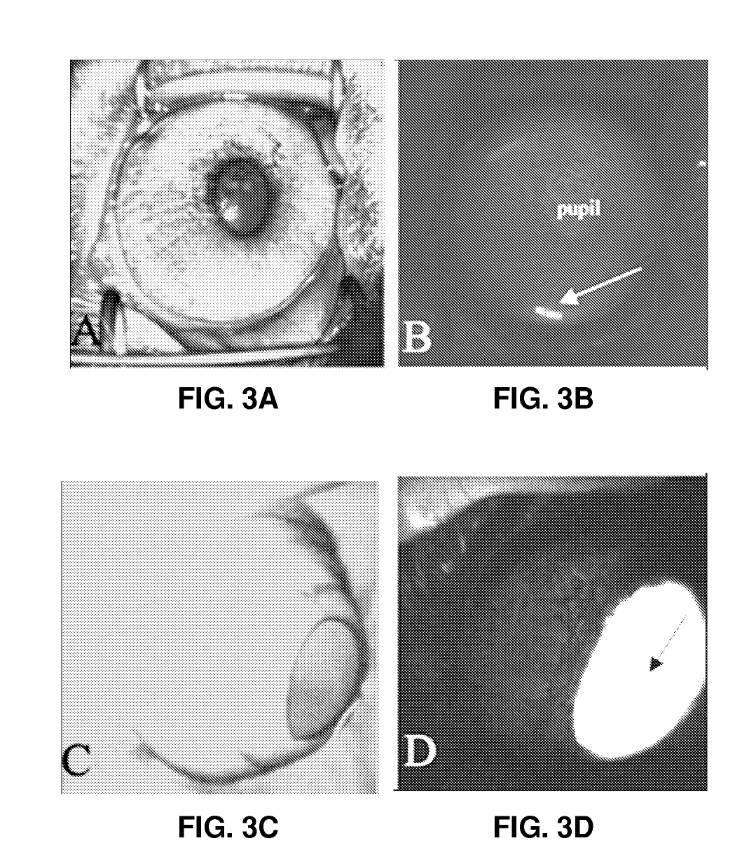

We determined that in the anterior chamber of the eye there are vertical upwards convection currents flowing from the 6 o'clock position to the 12 o'clock position driven by the higher temperatures of the aqueous humor in contact with the iris. We also determined that in the anterior chamber there are downward convection currents of aqueous flow proceeding from the 12 o'clock position to the 6 o'clock position driven by the cooler temperatures of aqueous humor adjacent to the corneal endothelium. See FIG. 2. We hypothesized that these aqueous humor currents can effectively carry anti-hypertensive drugs throughout and around the anterior chamber in a 360 degrees distribution pattern if the delivery system releases drug directly into the aqueous humor and we demonstrated the effectiveness of the convection currents distributing a surrogate drug in the anterior chamber with imaging studies of release from a sustained-release implant placed in the anterior chamber. See FIG. 3.

Additionally, we made microspheres with diameters greater than 30 microns and with sufficient density to settle into the inferior angle of the anterior chamber following intracameral injection of the microspheres. Importantly, the greater than 30 micron diameter of the microspheres is such that the microspheres are not either cleared through or embedded in the trabecular meshwork, thereby ensuring that free drug is released directly into the aqueous currents to effectively distribute drug to the angle along a 360 degrees distribution pattern. Free drug can transit through the trabecular meshwork and the iris root into the ciliary body region. To accelerate settling of microsphere formulations to the 6 o'clock position, an uncrosslinked or cross-linked hydrogel, such as a hyaluronic acid or a methylcellulose compound can be added in a 0.2% to 4% concentration to the microspheres, as a carrier. The addition of the gel can facilitate passage of microspheres with diameters greater than 30 microns through small gauge (e.g. 27 to 30 G) needles and permits use in pre-filled syringes. Alternative delivery systems, such as solid implants with bioerodible polymers can also be used since they will settle at the 6 o'clock position following injection into the anterior chamber (see FIG. 3B).

Thus, FIG. 3 presents evidence of aqueous humor drug delivery (after intracameral placement of an implant at the 6 o'clock position) via convection currents visualized using a Heidelberg HRA imaging device. FIG. 3A is an external photograph of a rabbit eye in primary gaze. FIG. 3B is an image of the rabbit eye in 3A with fluorescein filters in place on the HRA. In FIG. 3B the rabbit is 2 days post-implantation of a sustained-release fluorescein implant in the anterior chamber and it can be seen that the implant has settled at the 6 o'clock position (arrow). FIG. 3C is an external photograph of the same rabbit eye rotated down.

FIG. 3D is an image of the rabbit eye in 3C with the HRA. In FIG. 3D the rabbit is 7 days post-implantation of a sustained-release fluorescein implant in the anterior chamber that has settled at the 6 o'clock position as shown in 3B. Note that with the convection currents, free fluorescein released from the implant has become evenly distributed throughout the anterior chamber (arrow) and will thereby have 360 degree exposure to the trabecular meshwork and ciliary body, the target tissues for anti-hypertensive treatment.

We also determined that microspheres or other sustained-release delivery systems that have a lower density than the aqueous humor can have therapeutic utility because they will float up and settle superiorly in the 12 o'clock position. Here the drug delivery system can release drug into the convection currents and this is a suitable alternative to delivery systems that are located at the 6 o'clock position in order to distribute free drug to the angle 360 degrees. Thus, we took time lapsed images of a drug surrogate injected at 12 o'clock position and demonstrated presence of anterior chamber convection currents which distributed the drug surrogate throughout (homogenous drug exposure over 360 degrees) the anterior chamber within 20 minutes.

As set forth below we developed a technique where the PVA stabilizer used in the manufacturing process was washed with water 5 times to strip the PVA component off the microspheres. This chemical modification allowed the microspheres to float up to the 12 o'clock position in the anterior chamber because microsphere surfaces become very hydrophobic after losing hydrophilic PVA and water cannot effectively wet particle surfaces. It is critical for the drug delivery system to rapidly settle inferiorly or superiorly to clear the visual axis of any obstruction.

Unexpectedly, pharmacokinetic studies examining drug tissue levels in the ciliary body demonstrated high levels following injection of sustained-release implants into the anterior vitreous region. It has been previously thought that most drugs injected into the vitreous cavity would be diffuse and/or be directed by various mechanisms to the back of the eye and eliminated through the retina and choroid. We carried out imaging and pharmacologic studies and placement of delivery systems in the anterior vitreous base and determined that delivery of anti-hypertensive drugs to the ciliary body can thereby be achieved with resultant lower IOP. These imaging studies demonstrated that drug placed into the anterior vitreous base can access the aqueous humor in the posterior chamber and rapidly disperse drug 360 degrees in both animal and human eyes. Drug delivery systems, such as microspheres and implants, can be routinely placed into the anterior vitreous using standard surgical procedures. The MRI imaging studies were with porcine eye following injection of a drug surrogate into the anterior vitreous. The drug passed rapidly into the posterior chamber and was distributed around the ciliary body in a 360 degrees pattern. Additionally, we carried out MRI imaging studies of a human eye following injection of a drug surrogate in the anterior vitreous and demonstrated that the drug passed rapidly into the posterior and anterior chamber, showing that vitreous injections can deliver drugs to the aqueous humor.

### Example 2

### Development of Sustained Release Microspheres

Introduction

In this Example we made and evaluated various hypertensive drug containing microspheres for use to treat glaucoma and related ocular conditions. Thus, we developed sustained release microspheres for treatment of ocular hypertension. The microspheres we made can provide from about 3 months to about 6 months of IOP reduction (as a monotherapy, that is without the need for supplemental anti-hypertensive drug containing eye drops) with very reduced corneal hyperemia (as compared to sub-tenon administration of the same microspheres or implant). The microspheres contain at least about 10 weight % hypertensive drug load and have a mean diameter of greater than 30 μm, as we determined that use of microspheres with a diameter greater than 30 μm reduces eye hyperemia after intracameral administration of the microspheres.

The microsphere manufacturing process was started with a solvent evaporation process using dichloromethane as a solvent and SDS surfactant. However, when latanoprost was incorporated, the process had numerous problems with very low yields, much smaller particle sizes, and poor drug entrap efficiencies. Therefore we developed process improvements by changing both the solvent and the surfactant. Eventually, we finalized the process with ethyl acetate as the solvent and 1% poly vinyl alcohol (PVA) as stabilizer. Also we were able to obtain hypertensive drug loading as high as 19 wt %. The microsphere diameters were maintained above 30 um, and can be made up to 65 um if reduced shear rates were used. Microsphere diameter and diameter distribution were determined using a Malvern Mastersizer 2000 instrument. Each sample was analyzed by average of 5 readings. Microsphere fractionations were also practiced by filtering through sieves to maintain minimum size cutoff. Many different PLA and PLGA polymers and polymer blends were screened to obtain a family of release profiles and to select candidates for in vivo microsphere administration. The in vitro release rate of the formulations studied ranged from 17 to 88 ug/day.

Extensive morphological studies were performed on the microspheres made. Thus microsphere surfaces were examined by SEM (using a Zeiss EVO 40 instrument), and the drug distribution inside the particle were determined by freeze facture SEM. Surface and internal morphology was examined using SEM freeze-dried microspheres which were dusted over double-sided adhesive graphite tape with the other side applied to an aluminum stub. Excess samples were removed and stub sputter coated with a 5-10 nm gold layer. Internal microsphere morphology was observed following microsphere freeze fracture carried out by applying monolayer microspheres on carbon tape, covered with another carbon tape on stub, and this sandwich structure was then submerged into liquid nitrogen for 10 seconds. The sandwiched monolayer was broken up to result into fractured microspheres.

Samples before and after drug release were compared, and drug loaded samples were also compared with placebos. They exhibit strikingly different morphologies, and revealed close relationships among polymer properties, morphologies, and release behaviors. In all twenty three different microsphere formulations we made. The two microsphere Formulation A and Formulation B were evaluated in vivo. We determined that these sustained release microsphere formulations A and B can release of anti-hypertensive gent over a several month period and that they the microspheres can be administrated by intraocular injection on an outpatient basis.

Formulation A Microspheres

Initially, we found that introduction of latanoprost into the polymer matrix significantly reduced the cohesion and resulted in small microparticle diameters. Additionally, poor drug entrap efficiencies (low wt % drug load) were attributed to much increased drug solubility in water due to SDS and long slow DCM evaporation process. DCM is not water miscible, and its evaporation process takes quite long time during which latanoprost has plenty of time to diffuse into water phase. We carried out experiments to decrease latanoprost aqueous solubility and to change the stabilizer used (from SDS to polyvinyl alcohol). Another process improvement was to gradually add more water miscible solvent, such as acertonitrile or ethyl acetate. This expedited particulate drying process. The final process used was a solvent extraction process.

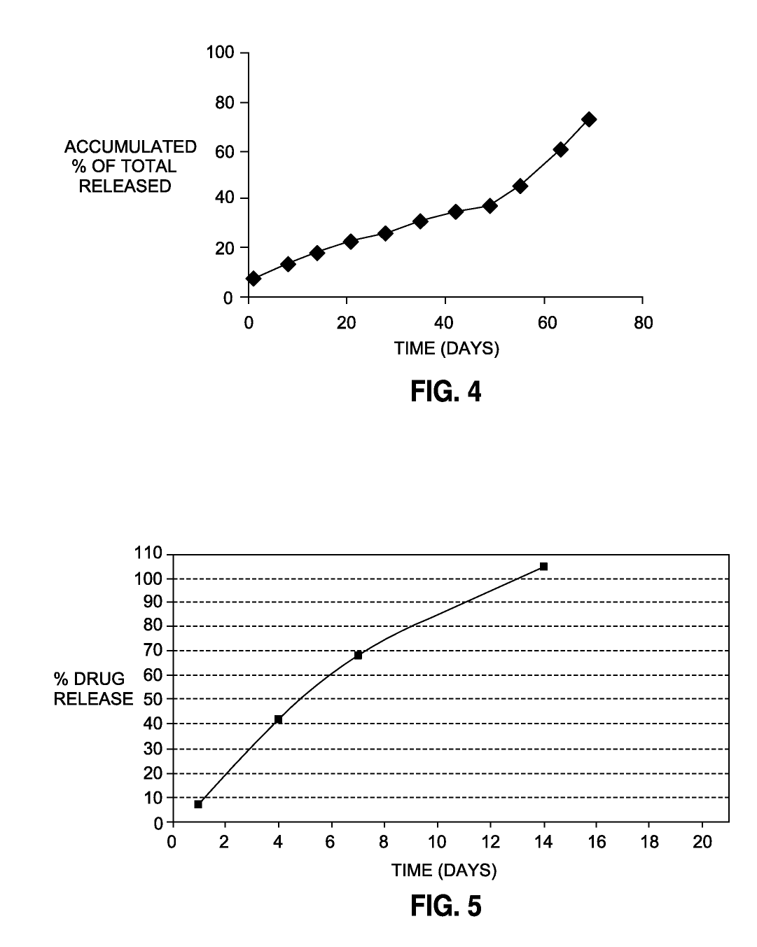

With these process improvements, Formulation A microspheres were made with the polymer 75:25 Poly(D,L, lactide-coglycolide)(Resomer RG755, Boehringer Ingelheim, Ingelheim, Germany) with a latanoprost content of 23.8%. Latanoprost (200 mg), a viscous oil at room temperature, and the polymer (600 mg) were dissolved in 5.6 ml ethyl acetate. This solution was added to 160 ml 1% PVA water via a micro-pipette while shearing. The mixture at 3000 rpm for 5 min with a Silverson homogenizer. After shearing, the milky white emulsion was mildly agitated in a hood for 3-5 hrs to allow solvent evaporation. The suspension was passed through 106 um and 34 um sieves to remove any fractions bigger than 106 um and smaller than 34 um. The supernatant was removed by centrifuging the suspension at 2000 rpm for 15 min, and 10 ml DI water was added to reconstitute the microspheres. The microsphere suspension was lyophilized to obtain free flowing dry powder. The vehicle used to suspend the microspheres before injection was 2% CMC and 0.1% wt Tween 80 (polysorbate 80) in 0.9% saline. The mean microsphere diameter size was about 60 um. The in vitro release rate (from a 50 ul dose of 20% microspheres) in a PBS medium with 0.1% Triton X-100 [octylphenol polyethoxylate]) of the latanoprost from the Formulation A microspheres was with zero order (constant amount of drug released per unit time) release kinetics over a significant period of time, as shown by FIG. 4. The Formulation A microspheres showed in vitro release rates of about 21 ug/day for the first 2 weeks.

Typically a sustained release drug delivery system releases incorporated drug following first order release kinetics, by which initial high levels of drug are released followed by a decrease (often an exponential decrease) in the drug release rate. Such a variable rate of drug dosing (over dose followed by under dose) to a target tissue is suboptimal for therapeutic treatment of an ocular condition. On the other hand first order drug release is optimal and is a highly beneficial dosing regime for successful treatment of intraocular tissues.

The microspheres were suspended in above mentioned vehicle at 20% concentration. The resulting suspension was injected through the scleral into the anterior chamber or into the vitreous through a 25 G needle. Alternatively, the microspheres can be suspended in a variety viscous gels, and they can be injected with as small as a 30 G needle. Two to ten milligrams microspheres were injected into dog eyes and saw significant IOP (50% from baseline) decrease for a period of time greater than 5 weeks. The microspheres can be injected into different sections of an eye including intracameral, intravitreal, and subtenon. The release rates can be adjusted by using different doses of the microspheres. The microspheres can also be injected with an applicator that allows for a ‘dry’ injection with or without the use of an aerosol. Here, the microspheres without the use of a wet vehicle can be injected into the anterior chamber or vitreous cavity without appreciably increasing the volume of the compartment. The Formulation A microsphere formulation has the potential to release latanoprost for between 2 to 7 months with a single intracameral or intravitreal injection.

Formulation B Microspheres

Formulation B microspheres were made with Resomer R203H (poly-DL-lactic acid) (“PLA”) with latanoprost content of 12.4%. The manufacturing process was similar to the Formulation A microsphere process set forth above. The microspheres were screened to isolate those with diameters equal to or greater then 34 microns and the resultant mean diameter was 45 microns. The in vitro release rates in PBS medium with 0.1% Triton X-100 (octylphenol polyethoxylate) exhibited near zero order release kinetics are shown in FIG. 5. The in vitro release rates of the Formulation B microspheres releasing was about 88 ug/day over a 2 week period.

We discovered that carrying out a further processing step upon the microspheres made provided the important feature of reducing an undesirable side effect upon in vivo administration of the microspheres. Thus, one side effect of microspheres injected into an eye can be corneal hyperemia. We determined that use of the two purification steps of size fractionation and washing prevented almost all hyperemia upon intraocular injection of the so further processed microspheres. These two steps were carried out by filtering the microsphere suspension through 34 um sieves to remove any smaller population of microspheres followed by washing the resulting microparticles were washed with water 3 to 5 times. When these purified microspheres were injected intracameral into dog eyes, there was significant (50 to 80% reduction) improvement in the corneal hyperemia observed.

Summary

Microspheres were successfully manufactured with a solvent extraction process. Homogenization shear rates and polymer concentrations were found to be the main factors for particle size control. The microsphere diameters can be varied from 1 um to up to 100 um, and fractionation with sieves produced well defined size ranges. Latanoprost loading can be optimized to about 25 wt %. Microspheres can be lyophilized without any protectant, and showed remarkable size stability. We found that e beam irradiation at moderate dosage (18 KGy) achieved excellent sterilization without any impact on subsequent drug release from the microspheres. A wide variety of release profiles were achieved mainly by using different polymer matrixes. The microspheres showed different morphology closely related to polymer properties and process conditions. Microsphere tends to settle fast and a high viscosity vehicle, for example 2% CMC, can slow down the settlement and make injection easier. Injections with 27 to 30 G needles can be obtained upon use of a suitable gel carrier.

Examples 3 to 7 below set forth in vivo (Beagle dogs) studies carried out by intraocular injection of microspheres or implant, suspended in aqueous vehicle (2% carboxy methylcellulose [CMC], 0.1% Tween 80 in 0.9% saline) or in a high viscosity, high shear rate gel (i.e. a suitable high molecular weight, polymeric hyaluronic acid). Microspheres or implant containing an anti-hypertensive agent was injected in one eye and placebo microspheres or implant was injected in the other eye as a control. IOP and hyperemia were monitored for multiweek periods. Ultra thin wall 25-27 gauge needles were used. When microspheres were used they were suspended in vehicle at 10% or 20% by weight.

### Example 3

### Intracameral Formulation A Microspheres

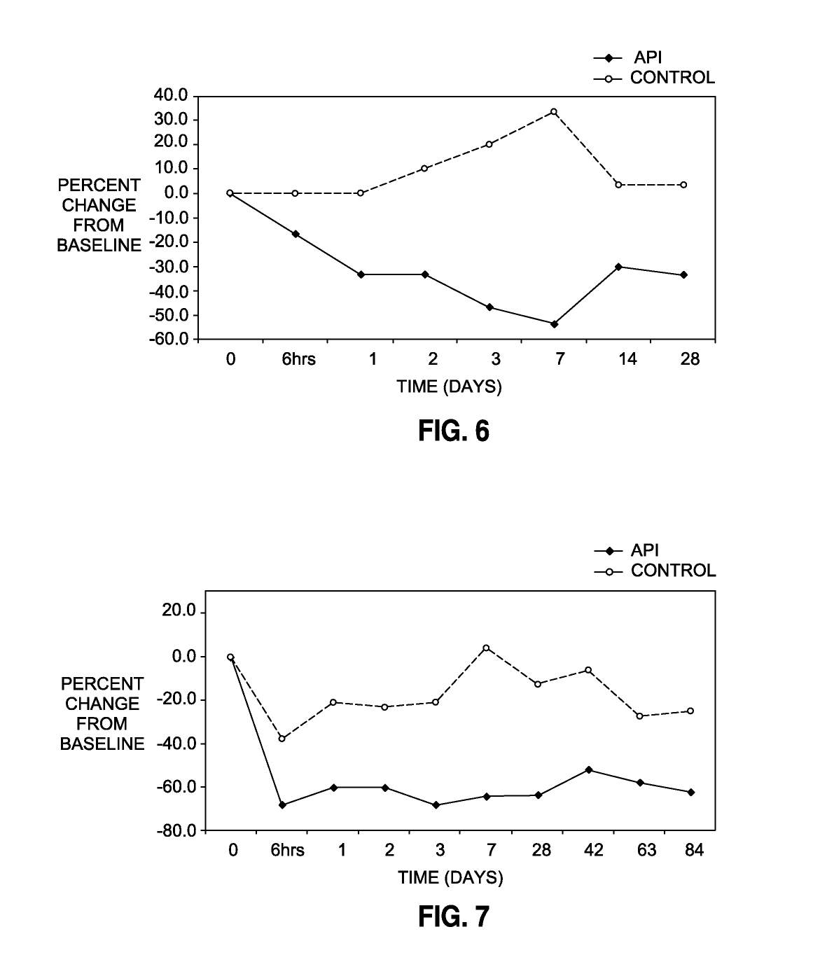

10 ul of Formulation A was injected into the left anterior chamber of a beagle dog using a shelving approach through the cornea with a 25 G hypodermic needle at the 12 o'clock position. The wound was self-sealing and the microspheres rapidly settled into the inferior angle within 30 minutes. FIG. 6 demonstrates a profound reduction in IOP in the left eye compared with the untreated right eye. This reduction in IOP was sustained for a number of weeks. As shown by FIG. 6, the left eye (solid line) received an intracameral injection of sustained-release latanoprost microspheres and IOP reduction of approximately 50% from baseline was recorded in the left eye by day 3 and an IOP reduction was sustained to at least the 1 month time point. No reduction of IOP was noted in the fellow control eye (dashed line) that received no injection. External photographs of the eye shows only mild conjunctival hyperemia.

### Example 4

### Intracameral Formulation B Microspheres

A dog received an intravitreal injection anteriorly with 50 μl of the Formulation B microsphere formulation in the left eye 4 mm behind the limbus in the supero-nasal quadrant, the right eye received an injection of placebo microspheres. In the left eye, the IOP was reduced to a maximum of about 40% below baseline and there was mild to moderate conjunctival hyperemia localized to the site of injection. The hyperemia grades in the opposite quadrants were consistently recorded as 0. There was no IOP reduction seen in the right eye that received the placebo microspheres.

### Example 5

### Intravitreal and Intracameral Bimatoprost Implant

Sustained-release bimatoprost heat extruded implants were made using 45 wt % resomer R203s (poly-DL-lactic acid), 20 wt % R202H (Poly (DL- lactide)), 30 wt % drug load, and 5 wt % PEG3350 as cosolvent. The total weight of the bar-shaped implants made were either 1.64 mg (492.4 ug drug load) or 800 μg, the latter, half size bar shaped implant measuring 1 mm wide and 2 mm long. The implants showed in vitro release of bimatoprost a over a four month period.

Intravitreal

One bimatoprost implant was inserted in the supero-nasal quadrant of the left dog eye anterior vitreous, 4 mm behind the surgical limbus. A placebo (no bimatoprost) implant was placed in the fellow eye. There was a significant reduction of IOP (up to about 45% below baseline) in the left vs. right eye). In addition, there was considerably less conjunctival hyperemia with intravitreal implant placement compared with sub-Tenon's placement.

Intracameral

One 800 μg bimatoprost implant was inserted into the anterior chamber through a shelved incision in clear cornea superiorly near the limbus of the right eye, the left eye received a placebo (no bimatoprost) implant. The implant was located superiorly at the 6 hour time point and settled inferiorly at the 6 o'clock position by 24 hours after insertion. There was slow bioerosion of the implant noted and no signs of intraocular toxicity. There was a large reduction in the IOP ranging from 60 to 70% below baseline recordings noted in the first 24 hours and maintained thereafter. See FIG. 7.

The implant released about 6 μg bimatoprost each day for the first 30 days after administration. The implant upon administration fit well into the well of the angle along the trabecular meshwork at the 6 o'clock position. The anterior chamber angle exists where the cornea meets the iris. At this location one find the trabecular meshwork which is the site where (in a normal eye) the aqueous humor drains out of the eye. If the aqueous humor cannot properly drain out of the eye, elevated intraocular pressure results. FIG. 7 is a graph of percent change from subject dog eye baseline intraocular pressure (y axis) against time in days (x axis) over the 84 day period after intracameral administration of the bimatoprost bar shaped implant, showing that an IOP drop of about 50% to 60% was maintained through the 84 day observation periods, showing great superiority of this single intracameral sustained release implant over the alternative daily (at least once each day for 84 days) anti-hypertensive agent eye drop administration to treat elevated IOP.

### Example 6

### Intracameral and Intravitreal EP2

The safety and tolerability of a single intracameral and intravitreal neat injection of the EP2 agonist Compound A (a molecule with a chiral center), was evaluated. The formulation used 0.1% of Compound A in normal saline. The chemical name of Compound A is 5-{(R)-1-[4-((S)-1-Hydroxy-hexyl)-phenyl]-5-oxo-pyrrolidin-2-ylmethoxymethyl}-thiophene-2-carboxylic acid isopropyl ester, its chemical formula is C₂₆H₃₅NO₅S and it's molecular weight is 473.63.

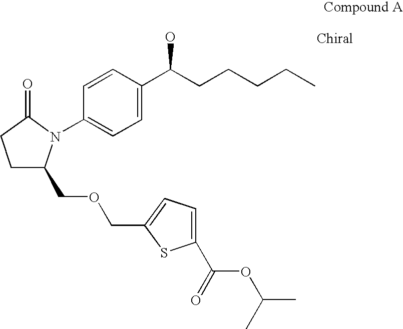

Compound A can also exist in the hydroxyl form:

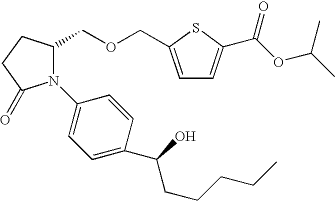

Dog 1: Intracameral Injection

A 50 microliter injection of Compound A formulation was performed into the anterior chamber of the left eye, the vehicle in the right eye, using a 27 G hypodermic needle. The IOP was reduced to a maximum of about 50% from baseline in the right eye. IOP reduction was maintained through day 3 compared to the fellow eye recordings. There was a maximum conjunctival hyperemia score of +0.5 present near the site of injection of both eyes in the first day, and subsequent recordings were all 0. There were no signs of ocular inflammation at the follow up examinations.

Dog 2: Anterior Vitreous Injection

A 50 microliter injection of the Compound A formulation was performed in the anterior vitreous of the left eye, entering just posterior to the ciliary body using a 27 G hypodermic needle. Vehicle was injected into the right eye. The IOP was reduced to a maximum of about 30% from baseline in the right eye. IOP reduction was maintained through day 3 compared to the fellow eye recordings. There was mild conjunctival hyperemia through day 3 in both eyes localized to the hemisphere where the injection occurred. There were no signs of ocular inflammation at the follow up examinations.

In summary, neat injections of Compound A in the anterior chamber and anterior vitreous were well tolerated and there was lowering of the IOP for a number of days following a single injection. In addition to reducing IOP an EP2 and EP4 agonist are can also be potent neuroprotective agent.

Compound A can be formulated in biodegradable polymeric microspheres or in a biodegradable polymeric implant, using the methods set forth in the Examples above, and administered intracameral or into the anterior vitreous to provide sustained anti-hypertensive (glaucoma treatment effect.

### Example 7

### Intracameral Latanoprost Implants

A sustained-release latanoprost (heat extrusion) implant comprising 30% latanoprost, 40% RG752s, 20% RG502s, 5% Plasdone and 5% PEG 3350 was made and intracameral injected into the left eye of a dog. A shelving incision was performed at the 11 o'clock position with a keratome and the latanoprost implant was inserted into the anterior chamber. The incision was closed using a 9-0 vicryl suture. There was about a 50% reduction of IOP from baseline recorded in this eye at the 24 hour time point.

### Example 8

### Formulation A Microspheres in Hyaluronic Acid

Formulation A microspheres with a drug content of 23.8% were mixed with a cross-linked hyaluronic acid (Juvederm). Using a gel-based microsphere suspension, a 27 G needle was used to inject the left eye with the active microsphere, the right eye with the placebo. The injection was facilitated by using the gel and there were no stoppages due to the needle becoming clogged. On day 1 post-injection, there was a 35% reduction in IOP in the left eye compared with baseline values. The microspheres appeared aggregated in the gel in the inferior angle and there was minimal ocular inflammation.

### Example 9

### Anti-Hypertensive Drug Containing Microspheres and Implants

Anti-hypertensive drug containing sustained release, biodegradable microspheres can be made for intracameral or anterior intravitreal injection to treat a hypertensive condition such as glaucoma. The anti-hypertensive drug can be one or more of EP2 agonists, such as Compounds A to O, including their salts, esters, prodrugs and derivatives. As noted below each of Compounds A to H and J has at least one chiral center. The microspheres can be made with the polymer 75:25 Poly(D,L, lactide-coglycolide)(Resomer RG755, Boehringer Ingelheim, Ingelheim, Germany) with a Compound (any of Compounds A to O) weight % content between 15 to 25 wt %. Thus 200 mg of a Compound (any of Compounds A to O) and 600 mg of the polymer are dissolved in about 6 ml ethyl acetate. This solution is then added to about 160 ml 1% PVA water via a micro-pipette while shearing. The mixture is then centrifuged at about 3000 rpm for 5 min with a Silverson homogenizer. After shearing, a milky white emulsion can be obtained which is mildly agitated in a hood for about 3 to 5 hrs to allow solvent evaporation. The suspension is then passed through 106 um and 34 um sieves to remove any fractions bigger than 106 um and smaller than 34 um. The supernatant is removed by centrifuging the suspension at 2000 rpm for 15 min, and 10 ml DI water is added to reconstitute the microspheres. The microsphere suspension is then lyophilized to obtain a free flowing dry powder. The vehicle used to suspend the microspheres before injection can be 2% CMC and 0.1% wt Tween 80 (polysorbate 80) in 0.9% saline.

Additionally, anti-hypertensive drug containing, sustained release, biodegradable implants can be made for intracameral or anterior intravitreal injection to treat a hypertensive condition such as glaucoma. The anti-hypertensive drug can be one or more of EP2 agonists, such as Compounds A to O. The implants can be made by hot-melt extrusion to contain about 30 wt % Compound (any of Compounds A to O), 40-60 wt % of a biodegradable poly (D,L-lactide-co-glycolide) polymer (Resomer® RG752s)(PLGA), 0-20% of a biodegradable poly (D,L-lactide) polymer (Resomer® R202s)(PLA), and 10% PEG-3350.

The Compound containing bioerodible polymer implants in this Example can be made by hot-melt extrusion using a mechanically driven ram microextruder but they can also be made by direct compression or solvent casting. The implants are preferably rod-shaped, but they can be made into any geometric shape by changing the extrusion or compression die. Polymers (the resomers) are used as received from Boehringer Ingelheim.

Compound (any of Compounds A to O) and polymer resomer powder are initially mixed (including 10 weight % PEG) using a spatula in a weigh-boat for 15 minutes. The mixture is then transferred into a stainless steel container containing two ¼″ stainless steel ball and mixing is continued using a Turbula mixer for two separate 15 minute cycles. The powder blend is mixed by hand using a spatula between each cycle and after the final cycle. The blended material is then compacted into an extruder barrel and the extruder barrel is placed into the heated well (between 50 and 55 degrees C.) of the piston extruder and extruded using 500 μm nozzle and a speed setting number of 0.0025. The extruded filament (rod shaped) implants are cut into one milligram implant (approximately 3 mm long). These sustained release, biodegradable implants can be administered intracameral to provide from 1 to 6 months (or longer) reduced IOP (anti-hypertensive effect).

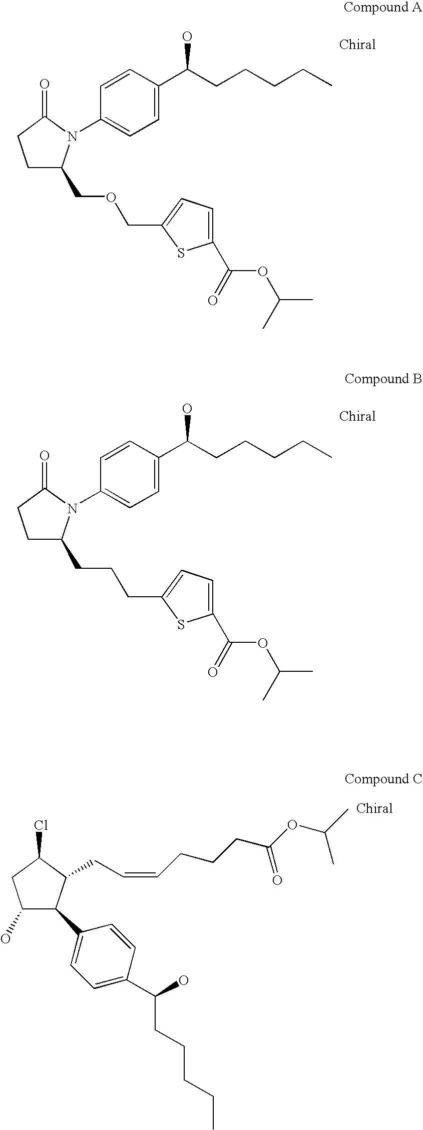

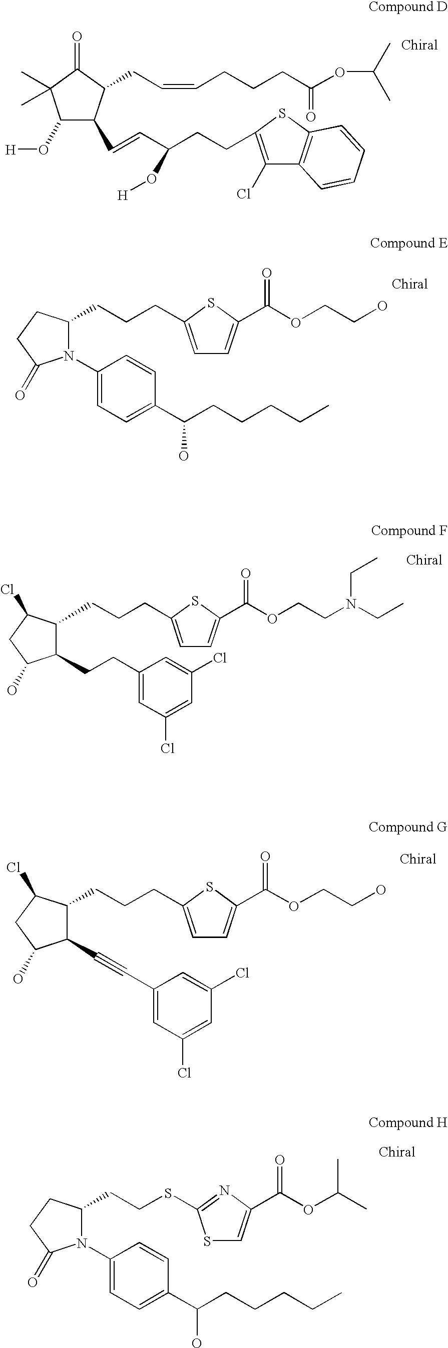

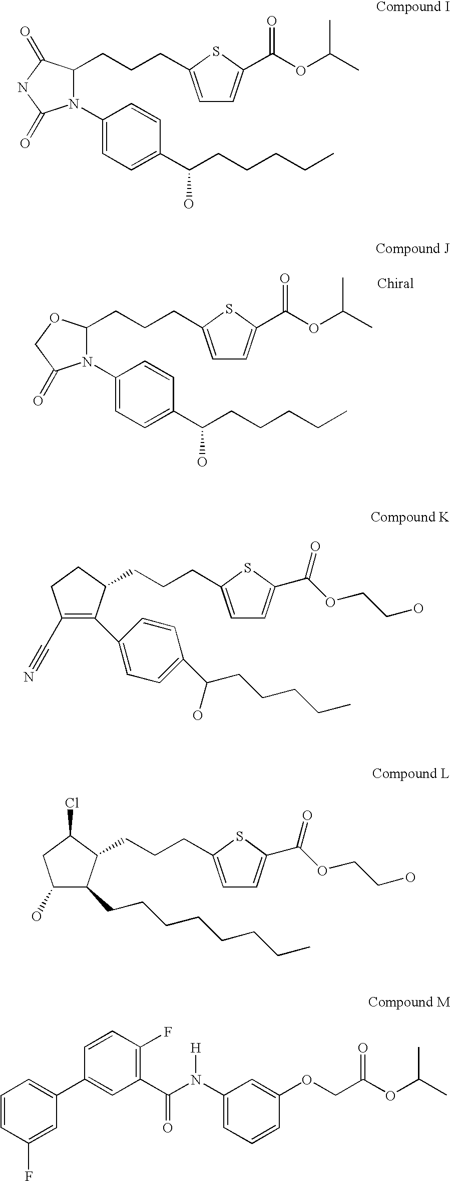

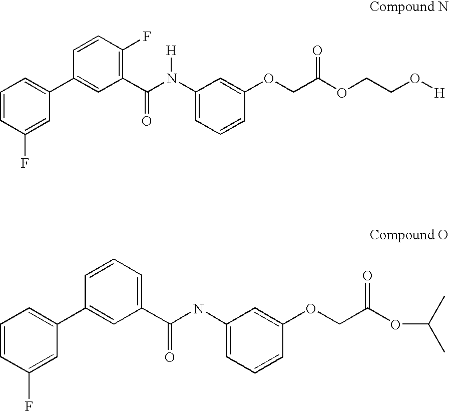

All references, articles, publications and patents and patent applications cited herein are incorporated by reference in their entireties.

While this invention has been described with respect to various specific examples and embodiments, it is to be understood that the invention is not limited thereto and that it can be variously practiced within the scope of the following claims.

We claim:

1. A method for treating elevated intraocular pressure, the method comprising the step of intracameral or anterior vitreal administration, to a patient with elevated intraocular pressure (IOP), of a sustained release implant comprising an anti-hypertensive agent and a biodegradable polymer, wherein the implant comprises from about 10 to about 50 weight percent the anti-hypertensive agent and from about 50 to about 90 weight percent the biodegradable polymer, and wherein the implant releases therapeutically effective amounts of the anti-hypertensive for a period of time between about 10 days and about 120 days.

2. The method of claim 1, wherein the implant can reduce IOP from about 20% to about 70% of baseline IOP.

3. A method for treating elevated intraocular pressure, the method comprising the step of intracameral administration against the trabecular meshwork, to a patient with elevated intraocular pressure, of a sustained release rod shaped implant comprising latanoprost or bimatoprost and a biodegradable polymer, wherein the implant comprises from about 10 to about 50 weight percent the latanoprost or bimatoprost and from about 50 to about 90 weight percent the biodegradable polymer, wherein the implant releases therapeutically effective amounts of the latanoprost or bimatoprost for a period of time between about 10 days and about 120 days.

4. A method for treating elevated intraocular pressure, the method comprising the step of intracameral or anterior vitreal administration to a patient with elevated intraocular pressure a plurality of sustained release biodegradable microspheres having an average diameter between 30 and 60 microns, the microspheres comprising from about 10 to about 30 weight percent an anti-hypertensive agent and from about 70 to about 90 weight percent a biodegradable polymer, wherein the microspheres release therapeutically effective amounts of the anti-hypertensive agent for a period of time between about 10 days and about 120 days.

5. The method of claim 4, wherein the biodegradable polymer comprises a polylactic polyglycolic copolymer (PLGA) and/or a poly lactic acid polymer (PLA).

6. The method of claim 4, wherein the antihypertensive agent is selected from the group consisting of latanoprost, bimatoprost and travoprost and their salts, esters and prodrugs.

7. The method of claim 4, wherein the antihypertensive agent is selected from the group consisting of Compounds A to O, and their salts, esters and prodrugs:

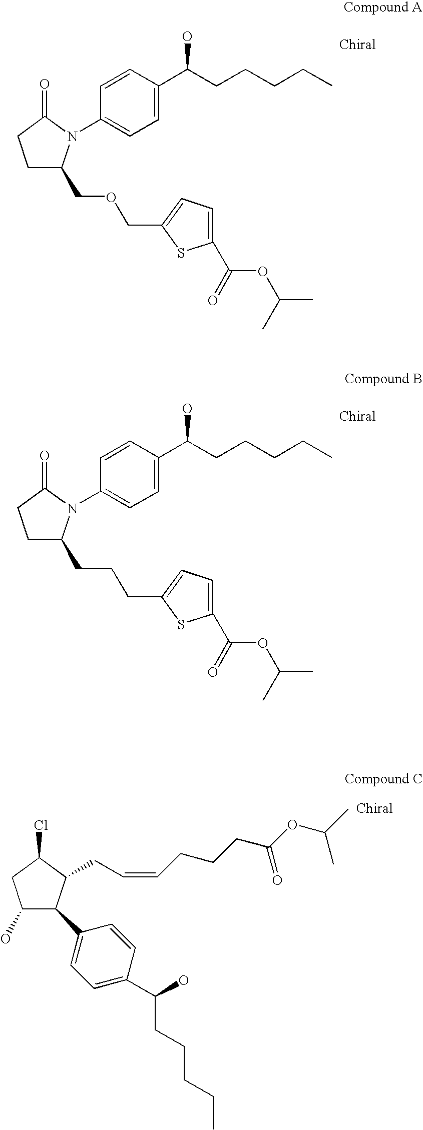

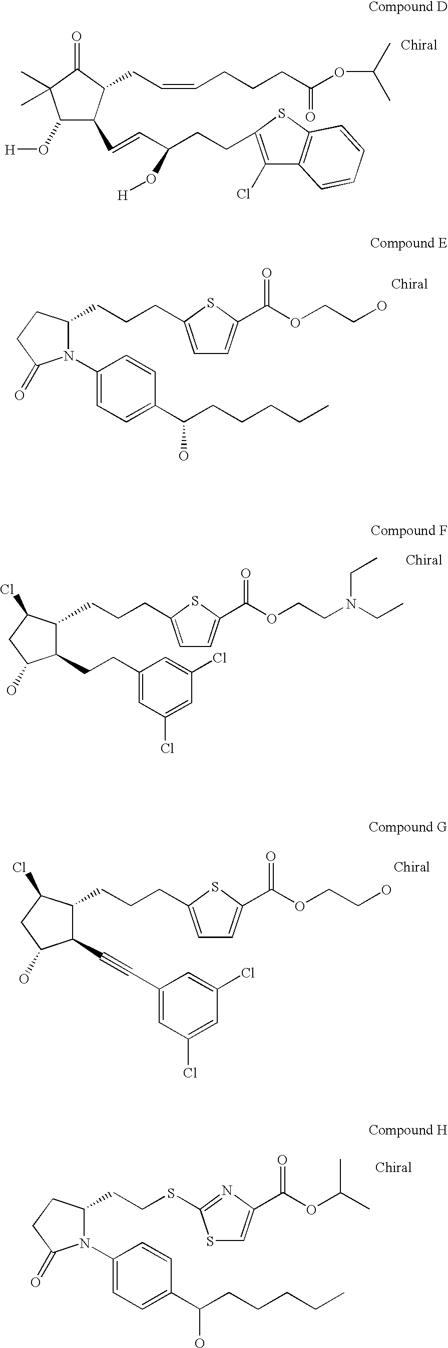

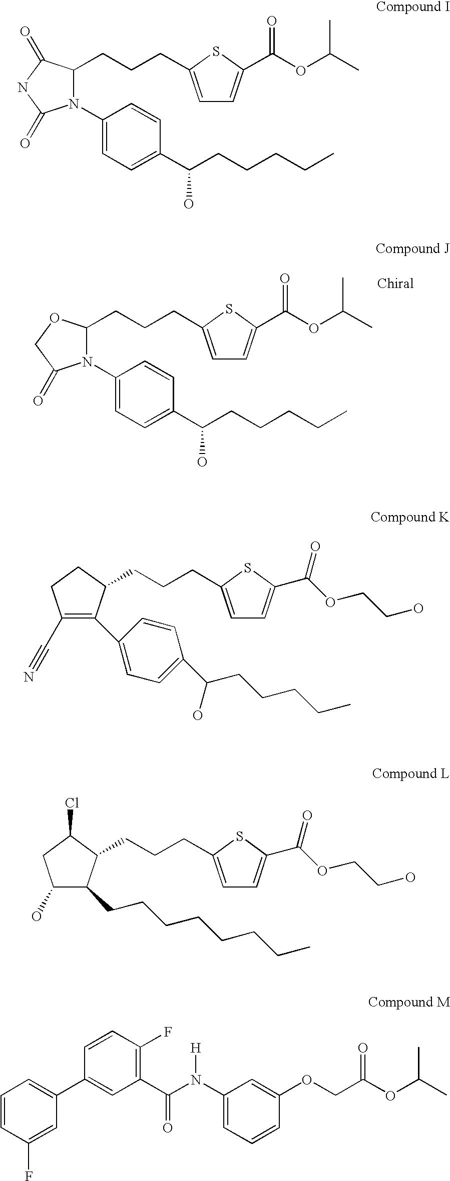

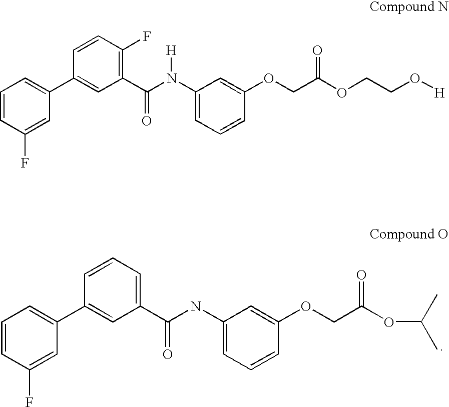

8. The method of claim 4, wherein the drug delivery system comprises a high viscosity hyaluronic acid.

9. A method for treating elevated intraocular pressure, the method comprising the step of intracameral administration to a patient with elevated intraocular pressure a plurality of sustained release biodegradable microspheres having an average diameter between 30 and 60 microns, the microspheres comprising from about 10 to about 30 weight percent latanoprost and from about 70 to about 90 weight percent a biodegradable polymer, wherein the microspheres release therapeutically effective amounts of the latanoprost for a period of time between about 10 day and about 120 days.

10. A method for treating elevated intraocular pressure, the method comprising the step of intracameral administration against the trabecular meshwork, to a patient with elevated intraocular pressure, a sustained release rod shaped implant comprising latanoprost and a biodegradable polymer, wherein the implant comprises from about 10 to about 50 weight percent an anti-hypertensive agent and from about 50 to about 90 weight percent a biodegradable polymer, wherein the implant releases therapeutically effective amounts of the latanoprost for a period of time between about 10 day and about 120 days.

11. A pharmaceutical composition for intraocular use to treat an ocular condition, the composition comprising a plurality of sustained release biodegradable microspheres having an average diameter between 30 and 60 microns, the microspheres comprising from about 10 to about 30 weight percent an anti-hypertensive agent and from about 70 to about 90 weight percent a biodegradable polymer, wherein the microspheres release therapeutically effective amounts of the anti-hypertensive agent for a period of time between about 10 day and about 120 days.

12. The composition of claim 11 wherein the microspheres comprise from about 1% to about 99% by weight of the polymer.

13. The composition of claim 11, wherein the polymer is a PLGA.

14. The composition of claim method of claim 11, wherein the antihypertensive agent is selected from the group consisting of latanoprost, bimatoprost and travoprost and their salts, esters and prodrugs.

15. The composition of claim 11, wherein the antihypertensive agent is selected from the group consisting of Compounds A to O, and their salts, esters and prodrugs:

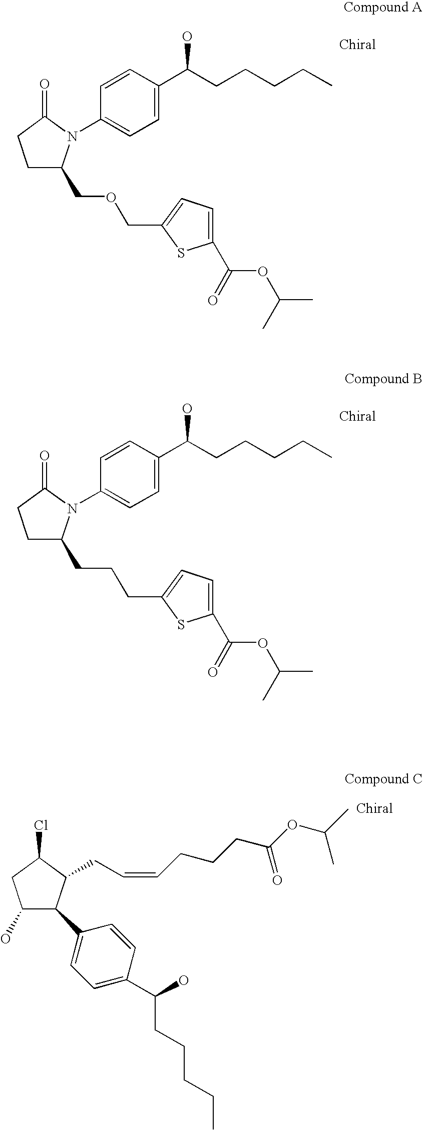

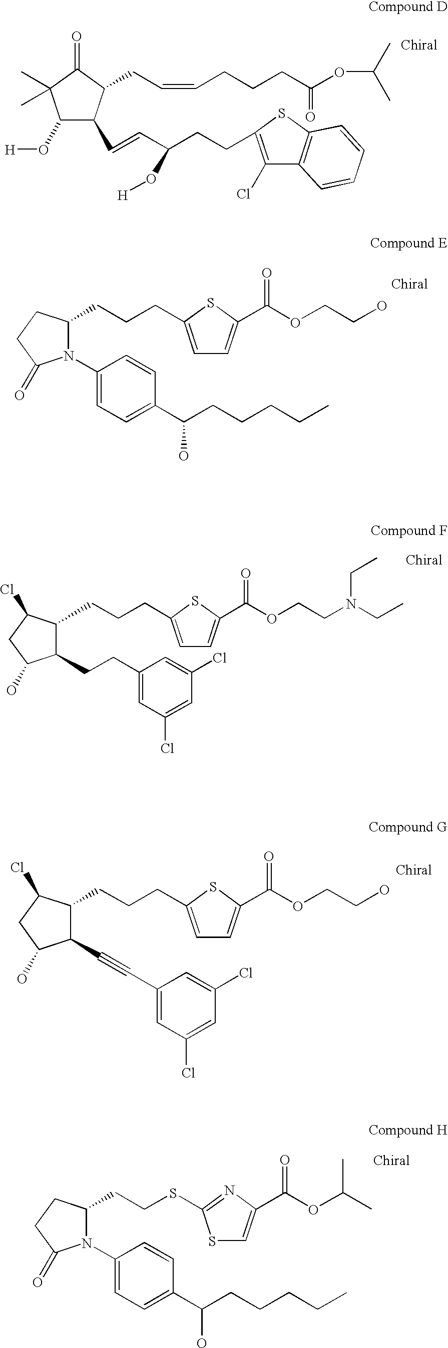

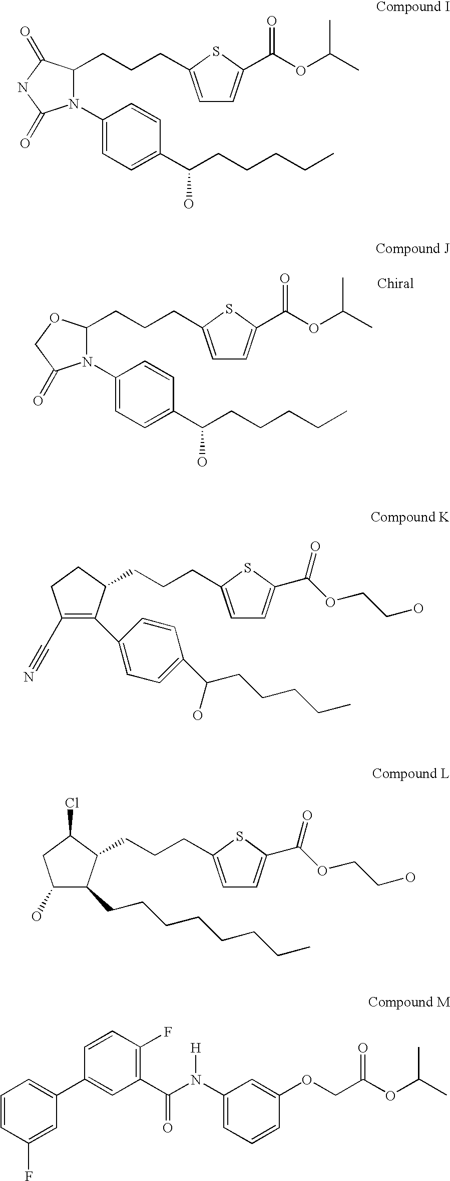

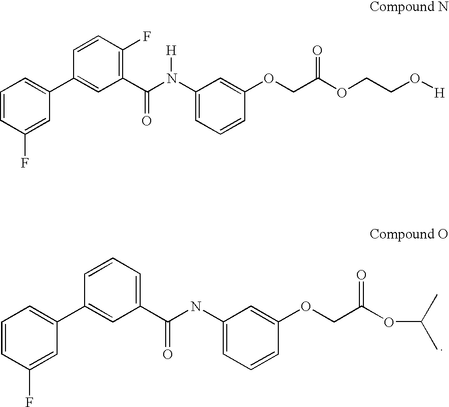

16. The composition of claim 11 further comprising a high viscosity hyaluronic acid.

17. The composition of claim 11 wherein the ocular condition is glaucoma.

18. The method of claim 1, wherein the antihypertensive agent is selected from the group consisting of Compounds A to O, and their salts, esters and prodrugs: 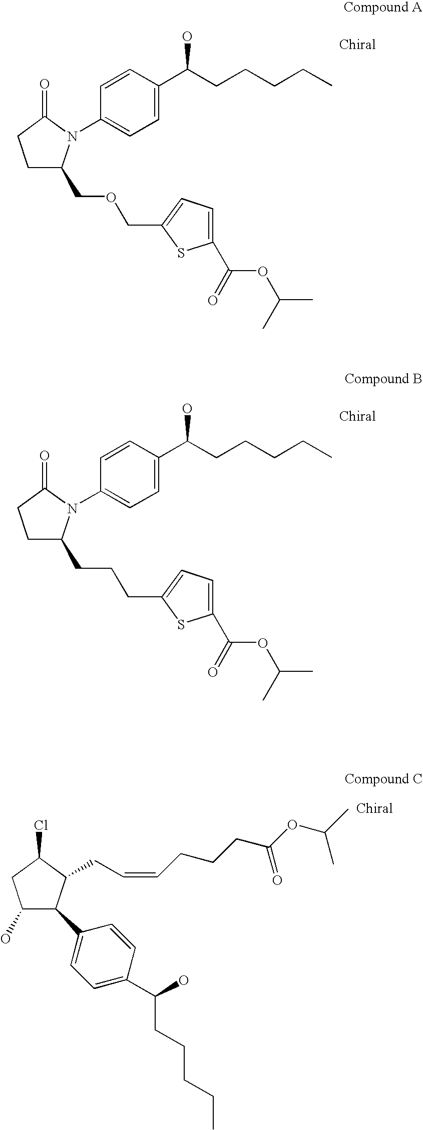 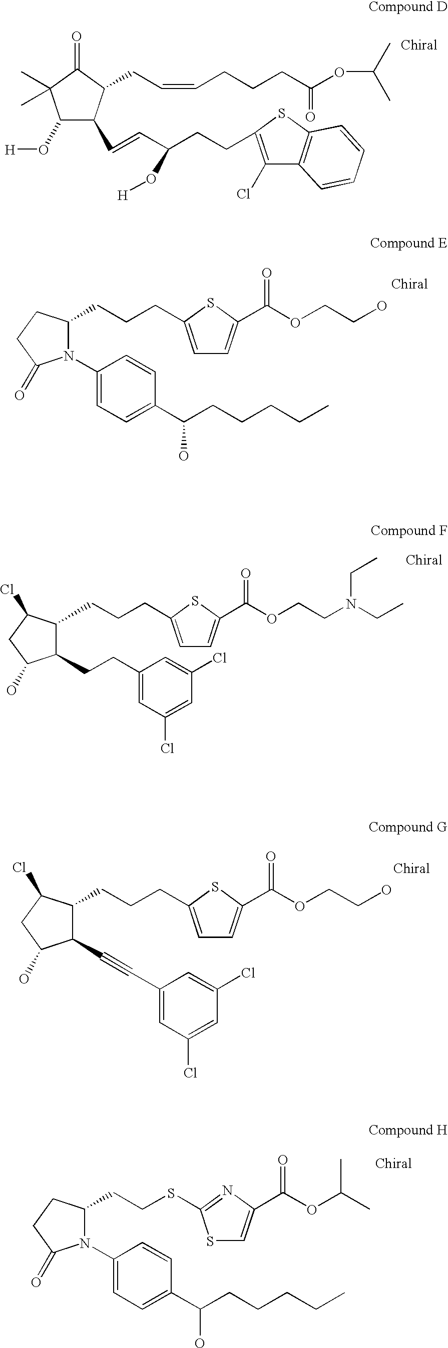 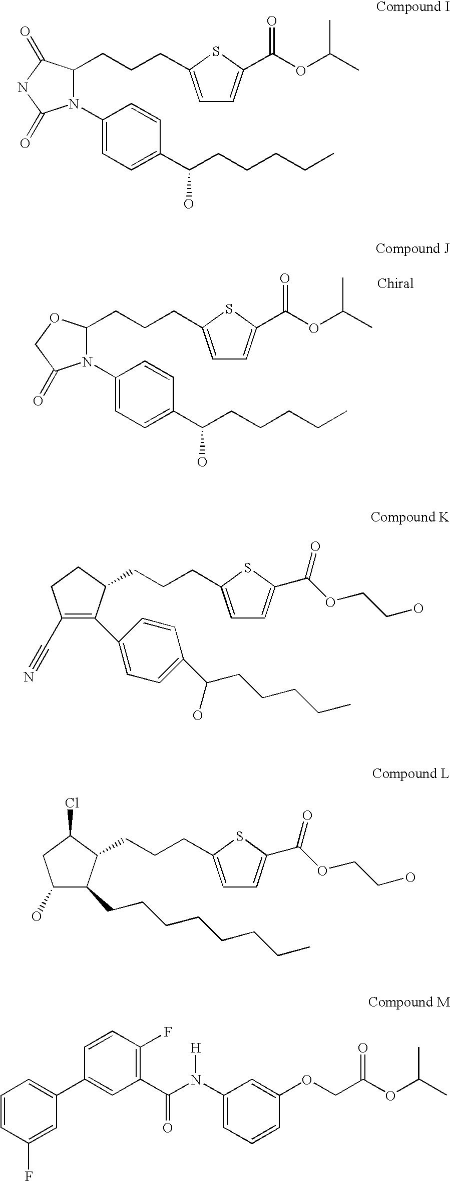 [chemical structure]
